SEQUENCE LISTING (8 sequences)

SEQ ID NO: 1 - DNA, 26 bp, Artificial Sequence
  Primer for Actb, F
tggaatcctgtggcatccatgaaacr

SEQ ID NO: 2 - DNA, 25 bp, Artificial Sequence
  Primer for Actb, F
taaaacgcagctcagtaacagtccg

SEQ ID NO: 3 - DNA, 23 bp, Artificial Sequence
  Primer for Il17, F
ctccagaaggccctcagactacr

SEQ ID NO: 4 - DNA, 18 bp, Artificial Sequence
  Primer for Il17, F
gggtcttcattgcggtgg

SEQ ID NO: 5 - DNA, 24 bp, Artificial Sequence
  Primer for Il17f, F
cccatgggattacaacatcactcr

SEQ ID NO: 6 - DNA, 18 bp, Artificial Sequence
  Primer for Il17f, F
cactgggcctcagcgatc

SEQ ID NO: 7 - DNA, 19 bp, Artificial Sequence
  Primer for Rorc, F
ccgctgagagggcttcacr

SEQ ID NO: 8 - DNA, 22 bp, Artificial Sequence
  Primer for Rorc, F
tgcaggagtaggccacattaca

(19) United States
(12) Patent Application Publication
(10) Pub. No.: US 2017/0298090 A1
(43) Pub. Date: Oct. 19, 2017

(54) Modulators of ROR-gamma Receptors, Composition and Use Thereof

(71) Applicant: KULING THERAPEUTICS, Beijing (CN)

(72) Inventors: Qiang DING, BEIJING (CN); Xiaohu Wang, Beijing (CN)

(21) Appl. No.: 15/481,591

(22) Filed: Apr. 7, 2017

Related U.S. Application Data
(60) Provisional application No. 62/319,855, filed on Apr. 8, 2016.

(51) Int. Cl. C07J 63/00; A61K 31/585; A61K 31/58; A61K 45/06; A61K 31/575

(52) U.S. Cl. CPC C07J 63/008; A61K 45/06; A61K 31/575; A61K 31/58; A61K 31/585

(57) ABSTRACT

The present invention provides novel methods to treat disease by modulating retinoid-related orphan receptor gamma (ROR-gamma) in vitro and in vivo with ursolic acid analogs, and compositions thereof. The methods and compounds disclosed herein are useful for inhibiting the differentiation of a population of T cells, or treating a disease related to Th17 cell responses in a subject. Examples of such diseases include, but are not limited to, autoimmune diseases, multiple sclerosis, rheumatoid arthritis, psoriasis and diabetes.

### FIELD OF INVENTION

The present invention relates to a novel method to treat diseases mediated by retinoid-related orphan receptor gamma (ROR-gamma) in vitro and in vivo. Further, the present invention relates to compounds and compositions for modulating ROR-gamma, and the preparation methods and uses thereof.

### BACKGROUND OF INVENTION

Autoimmune diseases occur when the immune system attacks and destroys healthy body tissue. Other inflammatory diseases, such as asthma, do not necessarily result from a direct attack on healthy tissue but rather from improper or uncontrolled immune responses. Agents that modulate the development and function of cells of the immune system can be useful as therapies for such diseases.

Th17 cells have recently emerged as a major player in the pathogenesis of multiple inflammatory and autoimmune disorders due to the production of IL-17 and IL-17F, two related cytokines with potent proinflammatory activities. (Korn, T., et al., IL-17 and Th17 Cells, Annu Rev Immunol. 27:485-517 (2009)). Functionally, Th17 cells play a role in host defense against extracellular pathogens by mediating the recruitment of neutrophils and macrophages to infected tissues. Differentiation of Th17 cells is controlled by a “master-regulator” transcription factor, ROR gamma t (RORγt), which directs a specific and heritable gene expression profile. Two isoforms of RORγ have been identified: RORγ1 and RORγt (also known as RORγ2). While RORγ1 is expressed in a variety of tissues including muscle, kidney, liver and thymus etc., RORγt is exclusively expressed in the cells of the immune system. Th17 cells have been shown to have key functions in mouse autoimmune disease models including experimental autoimmune encephalomyelitis (EAE) and collagen-induced arthritis (CIA). In addition, Th17 cells or their products have been shown to be associated with pathology of a variety of human inflammatory and autoimmune disorders, including multiple sclerosis, rheumatoid arthritis, psoriasis, Crohn's disease and asthma (Jetten Nucl. Recept. Signal. (2009) 7:e003. Manel et. Al. Na Immunol. (2008) 9:641-649). Currently, there is no effective therapy for controlling excessive Th17 cell responses and related diseases or disorders. Disclosed herein are ursolic acid and related compounds that are useful as modulators of ROR-gamma and further to control Th17 cells activities.

### SUMMARY OF THE INVENTION

The present invention generally provides a method to treat a variety of human inflammatory and autoimmune disorders, including but not limited to multiple sclerosis, rheumatoid arthritis, psoriasis, Crohn's disease, asthma, etc. Further, the present invention relates to the use of modulators of retinoid-related orphan receptor gamma (ROR-gamma) to modulate Th17 cells in vitro and in vivo. The present invention includes the compounds and compositions of said modulators, as well as the preparation methods and uses thereof.

In one aspect, the present invention provides a compound to modulate RORγt and Th17 cells in vitro and in vivo, which has the structure of Formula I:

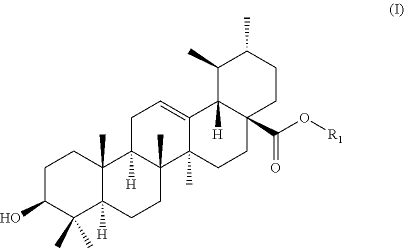

or a physiologically acceptable salt or hydrate or solvate thereof, wherein R₁ is independently selected from:

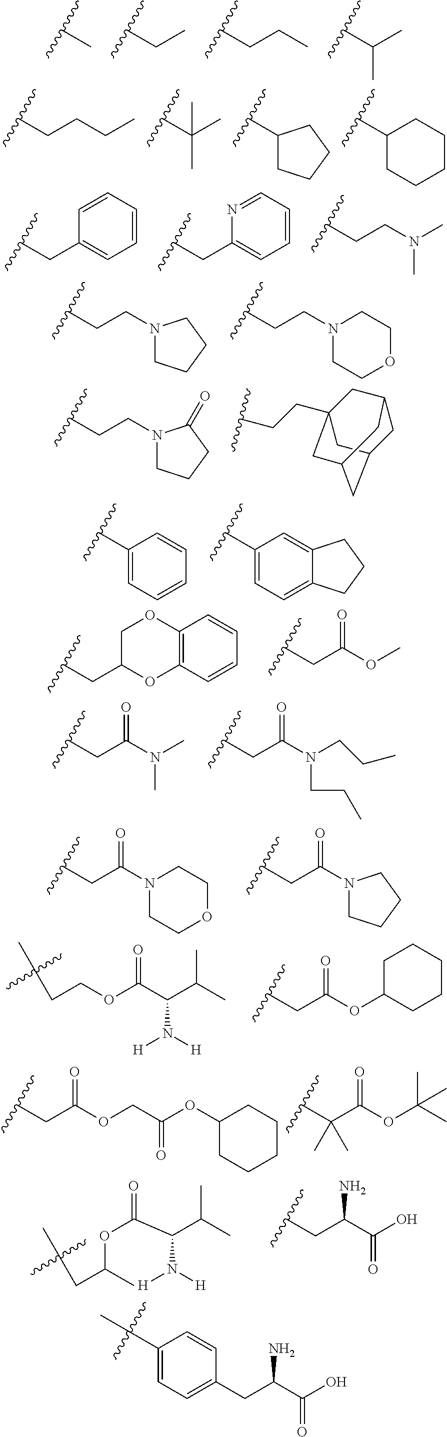

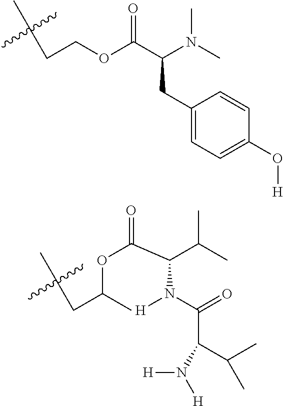 [SMILES: *CCOC(=O)[C@H](Cc1ccc(O)cc1)N(C)C ; *CC(C)OC(=O)[C@@H](NC(=O)[C@@H](N)C(C)C)C(C)C]

In another embodiment of the present invention, there is provided a compound of the formula II

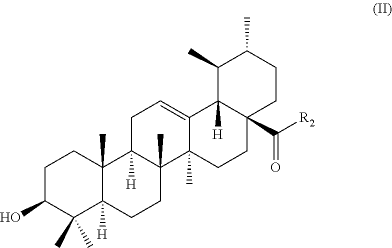

or pharmaceutically acceptable salt or hydrate or solvate thereof, wherein R₂ are independently selected from:

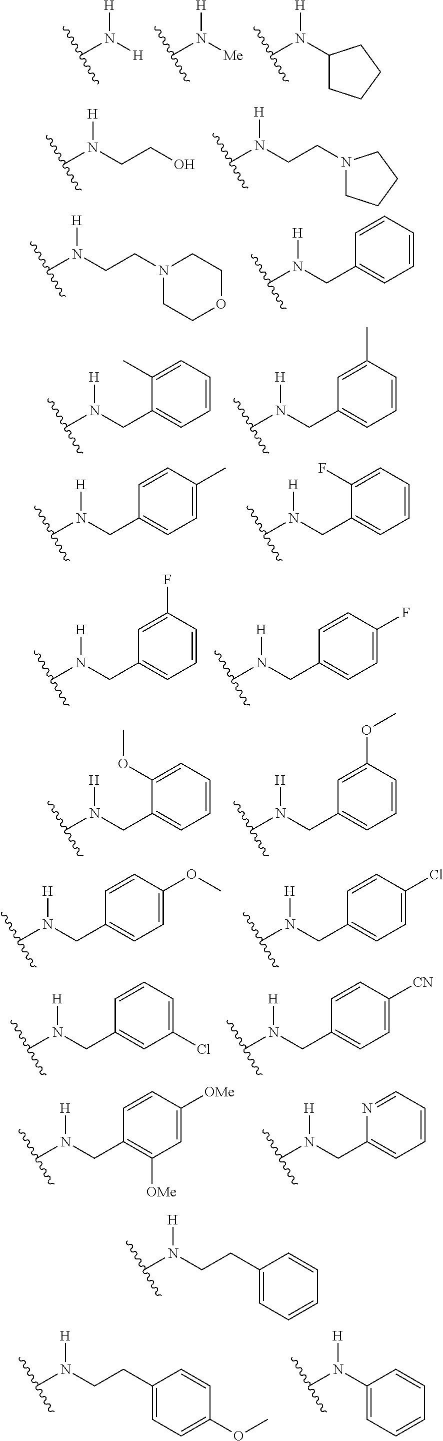

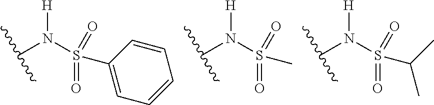

In another embodiment of the invention, there is provided a compound of the formula III

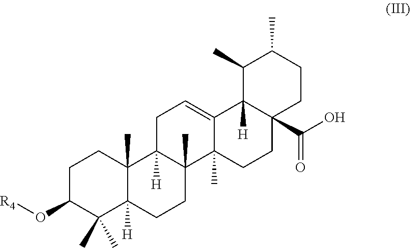

or pharmaceutically acceptable salt or hydrate or solvate thereof, wherein R₄ is independently selected from:

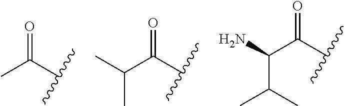

In one embodiment, example of the compound of the invention includes, but is not limited to
1. methyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
2. ethyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
3. propyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
4. isopropyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
5. butyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
6. tert-butyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
7. cyclopentyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
8. cyclohexyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
9. benzyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
10. pyridin-2-ylmethyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
11. 2-(dimethylamino)ethyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
12. 2-(pyrrolidin-1-yl)ethyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
13. 2-morpholinoethyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
14. 2-(2-oxopyrrolidin-1-yl)ethyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
15. 2-((3aR,5R)-octahydro-5H-2,5-methanoinden-5-yl)ethyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
16. phenyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
17. 2,3-dihydro-1H-inden-5-yl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
18. (2,3-dihydrobenzo[b][1,4]dioxin-2-yl)methyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
19. (5-methyl-2-oxo-1,3-dioxol-4-yl)methyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
20. 2-(dimethylamino)-2-oxoethyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
21. 2-(dipropylamino)-2-oxoethyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
22. 2-morpholino-2-oxoethyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
23. 2-oxo-2-(pyrrolidin-1-yl)ethyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
24. 2-methoxy-2-oxoethyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
25. 2-(cyclohexyloxy)-2-oxoethyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
26. 2-(2-(cyclohexyloxy)-2-oxoethoxy)-2-oxoethyl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
27. 1-(tert-butoxy)-2-methyl-1-oxopropan-2-yl (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate
28. O-((1S,2R,4aS,6aS,6bR,8aR,10S,12aR,12bR,14bS)-10-hydroxy-1,2,6a,6b,9,9,12a-heptamethyl-1,2,3,4,4a,5,6,6a,6b,7,8,8a,9,10,11,12,12a,12b,13,14b-icosahydropicene-4a-carbonyl)-D-serine
29. (R)-2-amino-3-(4-(((1S,2R,4aS,6a5,6bR,8aR,10S,12aR,12bR,14b5)-10-hydroxy-1,2,6a,6b,9,9,12a-heptamethyl-1,2,3,4,4a,5,6,6a,6b,7,8,8a,9,10,11,12,12a,12b,13,14b-icosahydropicene-4a-carbonyl)oxy)phenyl)propanoic acid
30. (1S,2R,4aS,6aS,6bR,8aR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxamide
31. (1S,2R,4aS,6aS,6bR,8aR,10S,12aR,12bR,14bS)-10-hydroxy-N,1,2,6a,6b,9,9,12a-octamethyl-1,3,4,5,6,6a,6b,7,8,8a,9,10,11,12,12a,12b,13,14b-octadecahydropicene-4a(2H)-carboxamide
32. (1S,2R,4aS,6aS,6bR,8aR,10S,12aR,12bR,14bS)-N-cyclopentyl-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxamide
33. (1S,2R,4aS,6aS,6bR,8aR,10S,12aR,12bR,14bS)-10-hydroxy-N-(2-hydroxyethyl)-1,2,6a,6b,9,9,12a-heptamethyl-1,3,4,5,6,6a,6b,7,8,8a,9,10,11,12,12a,12b,13,14b-octadecahydropicene-4a(2H)-carboxamide
34. (1S,2R,4aS,6aS,6bR,8aR,10S,12aR,12bR,14bS)-10-hydroxy-1,2,6a,6b,9,9,12a-heptamethyl-N-(2-(pyrrolidin-1-yl)ethyl)-1,3,4,5,6,6a,6b,7,8,8a,9,10,11,12,12a,12b,13,14b-octadecahydropicene-4a(2H)-carboxamide
35. (1S,2R,4aS,6aS,6bR,8aR,10S,12aR,12bR,14bS)-10-hydroxy-1,2,6a,6b,9,9,12a-heptamethyl-N-(2-morpholinoethyl)-1,3,4,5,6,6a,6b,7,8,8a,9,10,11,12,12a,12b,13,14b-octadecahydropicene-4a(2H)-carboxamide
36. (1S,2R,4aS,6aS,6bR,8aR,10S,12aR,12bR,14bS)-N-benzyl-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxamide
37. (1S,2R,4aS,6aS,6bR,8aR,10S,12aR,12bR,14bS)-10-hydroxy-1,2,6a,6b,9,9,12a-heptamethyl-N-(2-methylbenzyl)-1,3,4,5,6,6a,6b,7,8,8a,9,10,11,12,12a,12b,13,14b-octadecahydropicene-4a(2H)-carboxamide
38. (1S,2R,4aS,6aS,6bR,8aR,10S,12aR,12bR,14bS)-10-hydroxy-1,2,6a,6b,9,9,12a-heptamethyl-N-(3-methylbenzyl)-1,3,4,5,6,6a,6b,7,8,8a,9,10,11,12,12a,12b,13,14b-octadecahydropicene-4a(2H)-carboxamide
39. (1S,2R,4aS,6aS,6bR,8aR,10S,12aR,12bR,14bS)-10-hydroxy-1,2,6a,6b,9,9,12a-heptamethyl-N-(4-methylbenzyl)-1,3,4,5,6,6a,6b,7,8,8a,9,10,11,12,12a,12b,13,14b-octadecahydropicene-4a(2H)-carboxamide
40. (1S,2R,4aS,6aS,6bR,8aR,10S,12aR,12bR,14bS)-N-(2-fluorobenzyl)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxamide
41. (1S,2R,4aS,6aS,6bR,8aR,10S,12aR,12bR,14bS)-N-(3-fluorobenzyl)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxamide
42. (1S,2R,4aS,6aS,6bR,8aR,10S,12aR,12bR,14b5)-N-(4-fluorobenzyl)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxamide
43. (1S,2R,4aS,6aS,6bR,8aR,10S,12aR,12bR,14bS)-10-hydroxy-N-(2-methoxybenzyl)-1,2,6a,6b,9,9,12a-heptamethyl-1,3,4,5,6,6a,6b,7,8,8a,9,10,11,12,12a,12b,13,14b-octadecahydropicene-4a(2H)-carboxamide
44. (1S,2R,4aS,6aS,6bR,8aR,10S,12aR,12bR,14bS)-10-hydroxy-N-(3-methoxybenzyl)-1,2,6a,6b,9,9,12a-heptamethyl-1,3,4,5,6,6a,6b,7,8,8a,9,10,11,12,12a,12b,13,14b-octadecahydropicene-4a(2H)-carboxamide
45. (1S,2R,4aS,6aS,6bR,8aR,10S,12aR,12bR,14bS)-10-hydroxy-N-(4-methoxybenzyl)-1,2,6a,6b,9,9,12a-heptamethyl-1,3,4,5,6,6a,6b,7,8,8a,9,10,11,12,12a,12b,13,14b-octadecahydropicene-4a(2H)-carboxamide
46. (1S,2R,4aS,6aS,6bR,8aR,10S,12aR,12bR,14b5)-N-(4-chlorobenzyl)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxamide
47. (1S,2R,4aS,6aS,6bR,8aR,10S,12aR,12bR,14bS)-N-(3-chlorobenzyl)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxamide
48. (1S,2R,4aS,6aS,6bR,8aR,10S,12aR,12bR,14b5)-N-(4-cyanobenzyl)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxamide
49. (1S,2R,4aS,6aS,6bR,8aR,10S,12aR,12bR,14bS)-N-(2,4-dimethoxybenzyl)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxamide
50. (1S,2R,4aS,6aS,6bR,8aR,10S,12aR,12bR,14bS)-10-hydroxy-1,2,6a,6b,9,9,12a-heptamethyl-N-(pyrimidin-2-ylmethyl)-1,3,4,5,6,6a,6b,7,8,8a,9,10,11,12,12a,12b,13,14b-octadecahydropicene-4a(2H)-carboxamide
51. (1S,2R,4aS,6aS,6bR,8aR,10S,12aR,12bR,14bS)-10-hydroxy-1,2,6a,6b,9,9,12a-heptamethyl-N-phenethyl-1,3,4,5,6,6a,6b,7,8,8a,9,10,11,12,12a,12b,13,14b-octadecahydropicene-4a(2H)-carboxamide
52. (1S,2R,4aS,6aS,6bR,8aR,10S,12aR,12bR,14bS)-10-hydroxy-N-(4-methoxyphenethyl)-1,2,6a,6b,9,9,12a-heptamethyl-1,3,4,5,6,6a,6b,7,8,8a,9,10,11,12,12a,12b,13,14b-octadecahydropicene-4a(2H)-carboxamide
53. (1S,2R,4aS,6aS,6bR,8aR,10S,12aR,12bR,14bS)-10-hydroxy-1,2,6a,6b,9,9,12a-heptamethyl-N-phenyl-1,3,4,5,6,6a,6b,7,8,8a,9,10,11,12,12a,12b,13,14b-octadecahydropicene-4a(2H)-carboxamide
54. (1S,2R,4aS,6aS,6bR,8aR,10S,12aR,12bR,14bS)-10-acetoxy-1,2,6a,6b,9,9,12a-heptamethyl-1,3,4,5,6,6a,6b,7,8,8a,9,10,11,12,12a,12b,13,14b-octadecahydropicene-4a(2H)-carboxylic acid
55. (1S,2R,4aS,6aS,6bR,8aR,10S,12aR,12bR,14bS)-10-(isobutyryloxy)-1,2,6a,6b,9,9,12a-heptamethyl-1,3,4,5,6,6a,6b,7,8,8a,9,10,11,12,12a,12b,13,14b-octadecahydropicene-4a(2H)-carboxylic acid
56. (1S,2R,4aS,6aS,6bR,8aR,10S,12aR,12bR,14bS)-10-((D-valyl)oxy)-1,2,6a,6b,9,9,12a-heptamethyl-1,3,4,5,6,6a,6b,7,8,8a,9,10,11,12,12a,12b,13,14b-octadecahydropicene-4a(2H)-carboxylic acid
57. (1S,2R,4aS,6aS,6bR,10S,12aR,12bR,14bS)-10-hydroxy-1,2,6a,6b,9,9,12a-heptamethyl-N-(methylsulfonyl)-1,3,4,5,6,6a,6b,7,8,8a,9,10,11,12,12a,12b,13,14b-octadecahydropicene-4a(2H)-carboxamide (201509-1)
58. (1S,2R,4aS,6aS,6bR,10S,12aR,12bR,14bS)-10-hydroxy-N-(isopropylsulfonyl)-1,2,6a,6b,9,9,12a-heptamethyl-1,3,4,5,6,6a,6b,7,8,8a,9,10,11,12,12a,12b,13,14b-octadecahydropicene-4a(2H)-carboxamide (201509-2)
59. (1S,2R,4aS,6aS,6bR,10S,12aR,12bR,14bS)-10-hydroxy-1,2,6a,6b,9,9,12a-heptamethyl-N-(phenylsulfonyl)-1,3,4,5,6,6a,6b,7,8,8a,9,10,11,12,12a,12b,13,14b-octadecahydropicene-4a(2H)-carboxamide (201509-3)
60. 2-((L-valyl)oxy)ethyl (1S,2R,4aS,6aS,6bR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate (201702-3)
61. 2-((L-tyrosyl)oxy)ethyl (1S,2R,4aS,6aS,6bR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate (201702-4)
62. 2-((L-valyl)oxy)propyl (1S,2R,4aS,6aS,6bR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate (201702-1)
63. 2-((L-valyl-L-valyl)oxy)propyl (1S,2R,4aS,6aS,6bR,10S,12aR,12bR,14bS)-10-hydroxy-1,2,6a,6b,9,9,12a-heptamethyl-1,3,4,5,6,6a,6b,7,8,8a,9,10,11,12,12a,12b,13,14b-octadecahydropicene-4a(2H)-carboxylate (201702-2)

In another embodiment, the present invention provides a pharmaceutical composition comprising the compound of the present invention and at least one pharmaceutically acceptable carrier or diluent, wherein said compound is in free form or in a pharmaceutically acceptable salt form. Such composition may be an oral composition, injectable composition or suppository. And the composition may be manufactured in a conventional manner by mixing, granulating or coating methods.

In an embodiment of the invention, the composition is an oral composition and it may be a tablet or gelatin capsule. For example, the oral composition comprises the present compound together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets, together with c) binders, e.g., magnesium aluminum silicate, starch paste, gelatin, tragamayth, methylcellulose, sodium carboxymethylcellulose and or polyvinylpyrrolidone; and if desired, d) disintegrants, e.g., starches, agar, alginic acid or its sodium salt, or effervescent mixtures; and/or e) additives, e.g., absorbents, colorants, flavors and sweeteners.

In another embodiment of the invention, the composition is an injectable composition, and may be an aqueous isotonic solution or suspension.

In yet another embodiment of the invention, the composition is a suppository and may be prepared from fatty emulsion or suspension.

In one embodiment, the composition is sterilized and/or contains adjuvant. Such adjuvant can be preserving, stabilizing, wetting or emulsifying agent, solution promoter, salt for regulating the osmotic pressure, buffer and/or any combination thereof.

Alternatively or in addition, the composition may further contain other therapeutically valuable substances for different applications, like solubilizers, stabilizers, tonicity enhancing agents, buffers and/or preservatives.

In an embodiment of the invention, the composition may be a formulation suitable for transdermal application. Such formulation includes an effective amount of the compound of the present invention and a carrier. Preferably, the carrier may include absorbable pharmacologically acceptable solvents to assist passage through the skin of the host. A transdermal device contain the formulation may also be used. The transdermal device may be in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Otherwise, a matrix transdermal formulation may also be used.

In another embodiment of the invention, the composition may be a formulation suitable for topical application, such as to the skin and eyes, and may be aqueous solution, ointment, cream or gel well known in the art.

In another aspect, the present invention provides a method of inhibiting WNT secretion from a cell.

For therapeutically use, the compound of the present invention could be administered in a therapeutically effective amount via any acceptable way known in the art singly. As used herein, the therapeutically effective amount may vary widely depending on the severity of the disease, the age and relative health of the subject, the potency of the compound used and other factors. Generally, the satisfactory result is indicated to be obtained systemically at a daily dosage of about 100 to 500 mg/kg per body weight of the subject. In one embodiment, the indicated daily dosage for larger mammal as human is in the range from about 50 mg to about 1000 mg. In another embodiment, the compound is administered in divided doses up to four times a day or in retard form. In another embodiment, suitable unit dosage forms for oral administration comprise from 50 to 1000 mg active ingredient.

Alternatively, the compound of the present invention may be administered in a therapeutically effective amount as the active ingredient in combination with one or more therapeutic agents, such as pharmaceutical combinations. There may be synergistic effects when the compound of the present invention is used with a chemotherapeutic agent known in the art. The dosage of the co-administered compounds could vary depending on the type of co-drug employed, the specific drug employed, the condition being treated and so forth.

The compound of the present invention or the composition thereof may be administered by any conventional route. In one embodiment, it is administered enterally, such as orally, and in the form of tablets or capsules. In another embodiment, it is administered parenterally and in the form of injectable solutions or suspensions. In yet another embodiment, it is administered topically and in the form of lotions, gels, ointments or creams, or in a nasal or suppository form.

In another aspect, the invention also provides a pharmaceutical combination, for example, a kit, comprising a) a first agent which is the compound of the present invention as disclosed herein, in free form or in pharmaceutically acceptable salt form, and b) at least one co-agent. In addition, the kit may comprise instructions for its administration.

The combination of the present invention may be used in vitro or in vivo. In one embodiment, the desired therapeutic benefit of the administration may be achieved by contacting cell, tissue or organism with a single composition or pharmacological formulation that includes the compound of the present invention and one or more agents, or by contacting the cell with two or more distinct compositions or formulations, wherein one composition includes one agent and the other includes another. The agents of the combination may be administered at the same time or separately within a period of time. In one embodiment, the separate administration can result in a desired therapeutic benefit. The present compound may precede, be co-current with and/or follow the other agents by intervals ranging from minutes to weeks. A person skilled in the art could generally ensure the interval of the time of each delivery, wherein the agents administered separately could still be able to exert an advantageously combined effect on the cell, tissue or organism. In one embodiment, it is contemplated that one may contact the cell, tissue or organism with two, three, four or more modalities substantially simultaneously as the candidate substance, i.e., with less than about one minute. In another embodiment, one or more agents may be administered about between 1 minute to 14 days. In another embodiment, one or more agents may be administered about between 4 hours to 2 days.

In another aspect, the present provides a process for preparing the compound of the present invention or the salts or derivatives thereof.

In one embodiment, the compound having Formula (I) may be prepared following any one of the synthetic methodologies described in Examples below. In the reactions described, reactive functional groups, for example hydroxy, amino, imino, thio or carboxy groups, where these are desired in the final product, may be protected to avoid their unwanted participation in the reactions. Conventional protecting groups may be used in accordance with standard practice (see e.g., T. W. Greene and P. G. M. Wuts in “Protective Groups in Organic Chemistry”, John Wiley and Sons, 1991). Suitable leaving groups for use in the synthetic methodologies described include halogen leaving groups and other conventional leaving groups known in the art. For example, the leaving group is chloro or bromo.

In another embodiment, the compound of the invention or the salts thereof may also be obtainable in the form of hydrates, or their crystals may include for example the solvent used for crystallization (present as solvates). Salts can usually be converted to compounds in free form by treating with suitable basic agents, for example, with alkali metal carbonates, alkali metal hydrogen carbonates, or alkali metal hydroxides, potassium carbonate or sodium hydroxide. A compound of the invention in a base addition salt form may be converted to the corresponding free acid by treating with a suitable acid, such as hydrochloric acid. In view of the close relationship between the novel compounds in free form and those in the form of their salts, including those salts that may be used as intermediates, for example in the purification or identification of the novel compounds, any reference to the free compounds is to be understood as referring also to the corresponding salts, as appropriate.

Salts of the present compound with a salt-forming group may be prepared in a manner known in the art. Acid addition salts of compound of Formula (I) may thus be obtained by treatment with an acid or with a suitable anion exchange reagent. Pharmaceutically acceptable salts of the compound of the invention may be formed as acid addition salts from compound of Formula (I) with a basic nitrogen atom with organic or inorganic acids.

In one embodiment, suitable inorganic acids include, but are not limited to, halogen acids, such as hydrochloric acid, sulfuric acid, or phosphoric acid. In another embodiment, suitable organic acids include, but are not limited to, carboxylic, phosphoric, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4 aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4 methylbenzenesulfonic acid, methylsulfuric acid, ethylsulfuric acid, dodecylsulfuric acid, N cyclohexylsulfamic acid, N-methyl-, N-ethyl- or N-propyl-sulfamic acid, or other organic protonic acids, such as ascorbic acid.

Alternatively, it is also possible to use pharmaceutically unacceptable salts for isolation or purification, for example picrates or perchlorates. But for therapeutic use, only pharmaceutically acceptable salts or free compounds are employed, where applicable in the form of pharmaceutical preparations.

In yet another embodiment, compound of the present invention in unoxidized form may be prepared from N-oxides of compound of the invention by treating with a reducing agent in a suitable inert organic solvent at 0 to 80° C. In one embodiment, the reducing agent is sulfur, sulfur dioxide, triphenyl phosphine, lithium borohydride, sodium borohydride, phosphorus trichloride, tribromide, or the like. In another embodiment, the invert organic solvent is acetonitrile, ethanol, aqueous dioxane, or the like.

In yet another embodiment, prodrug derivatives of the compound of the present invention may be prepared by methods known in the art (for further details see Saulnier et al., (1994), Bioorganic and Medicinal Chemistry Letters, Vol. 4, p. 1985). In one embodiment, an appropriate prodrug may be prepared by reacting a non-derivatized compound of the invention with a suitable carbamylating agent such as 1,1-acyloxyalkylcarbanochloridate, para-nitrophenyl carbonate, or the like.

In yet another embodiment, protected derivatives of the compound of the present invention may be made by means known in the art. A detailed description of techniques applicable to the creation of protecting groups and their removal may be found in T. W. Greene, “Protecting Groups in Organic Chemistry”, 3rd edition, John Wiley and Sons, Inc., 1999.

In yet another embodiment, the present invention also includes isotopically-labeled compounds of the present invention, wherein one or more atoms are replaced by one or more atoms having specific atomic mass or mass numbers. Examples of isotopes that can be incorporated into compounds of the invention include, but are not limited to, isotopes of hydrogen, carbon, nitrogen, oxygen, fluorine, sulfur, and chlorine (such as ²H, ³H, ¹¹C, ¹³C, ¹⁴C, ¹⁵N, ¹⁷O, ¹⁸O, ³⁵S, ¹⁸F, ³⁶Cl). Isotopically-labeled compounds of the present invention are useful in assays of the tissue distribution of the compounds and their prodrugs and metabolites; examples of isotopes for such assays include ³H and ¹⁴C. In addition, in certain circumstances substitution with heavier isotopes, such as deuterium (²H or D), can provide increased metabolic stability, which offers therapeutic advantages such as increased in vivo half-life or reduced dosage requirements. Isotopically-labeled compounds of this invention can generally be prepared according to the methods described herein by substituting an isotopically-labeled reagent for a non-isotopically labeled reagent.

In yet another embodiment, compound of the present invention may be prepared as their individual stereoisomers. The process includes reacting a racemic mixture of the compound with an optically active resolving agent to form a pair of diastereoisomeric compounds, separating the diastereomers and recovering the optically pure enantiomers. Resolution of enantiomers may be carried out using covalent diastereomeric derivatives of the compound of the present invention, or by using dissociable complexes such as crystalline diastereomeric salts. Diastereomers have distinct physical properties presented by melting points, boiling points, solubilities, reactivity, etc., and may be readily separated by taking advantage of these dissimilarities. The diastereomers may be separated by fractionated crystallization, chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture may be found in Jean Jacques, Andre Collet, Samuel H. Wilen, “Enantiomers, Racemates and Resolutions”, John Wiley And Sons, Inc., 1981.

In conclusion, the compound of the present invention could be made by the process described below; optionally a pharmaceutically acceptable salt may be converted from the compound of the present invention; optionally a pharmaceutically acceptable N-oxide may be converted from an unoxidized form of the compound the present invention; optionally an individual isomer of the compound of the present invention is resolved from a mixture of isomers; and optionally a pharmaceutically acceptable prodrug derivative may be converted from a non-derivatized compound of the present invention.

Insofar as the production of the starting materials is not particularly described, the compounds are known or can be prepared analogously to methods known in the art or as disclosed in the examples hereinafter. One of skill in the art will appreciate that the above transformations are only representative of methods for preparation of the compounds of the present invention, and that other well-known methods can similarly be used.

In another aspect, the present invention also provides the use of the compounds disclosed herein or the compositions thereof in the preparation of medicines for treating metabolic disorders, such as obesity and diabetes.

Insofar as the production of the starting materials is not particularly described, the compounds are known or can be prepared analogously to methods known in the art or as disclosed in the Examples hereinafter. One of skill in the art will appreciate that the above transformations are only representative of methods for preparation of the compounds of the present invention, and that other well-known methods can similarly be used.

In one embodiment, methods for treating diseases and disorders related to Th17 cell responses in a patient in need of such treatment are provided herein. The described methods comprise the step of administrating to a patient in need thereof a therapeutically effective amount of an inhibitor of RORγt (also referred to herein as a “RORγt inhibitor”). Also, provided are methods of inhibiting the differentiation of TH17 cells, the expression of IL-17, IL-17F, IL-21 and IL-22 and/or the production of IL-17, IL-17F, IL-21 and IL22. Each of the methods comprises the step of administrating to a patient in need thereof a therapeutically effective amount of a RORγt inhibitor. The RORγt inhibitors taught herein include, but are not limited to, ursolic acid (“UA”), KL001 and other UA derived analogs such as those presented below in the examples. The present disclosure also contemplates RORγt inhibitors for the use in the manufacture of a medicament for the treatment of a disease or condition ameliorated by the inhibition of RORγt and IL-17 expression.

As described herein, UA, KL001 and related chemicals, including but not limited to the analogs provided in Examples 1 to 24 below, can selectively and effectively blocked the function of RORγt and associated immune responses in both differentiated and developing Th17 cells. As a result, these small molecules are useful to modulate the development and differentiation of Th17 cells, the expression of IL-17, IL-17F, IL21 and IL22 and/or the production of IL-17, IL-17F, IL21 and IL22 and treat disease and/or disorders associated with the same.

### BRIEF DESCRIPTION OF THE SEVERAL VIEWS OF THE DRAWINGS

FIG. 1 shows that UA, KL001 and other UA analogs inhibited IL-17 and IL-17F expression in Th17 cells.

FIG. 2 shows that KL001 dose-dependently inhibited Th17 differentiation with an IC50 value around 0.49 μM.

FIG. 3 shows that UA and KL001 ameliorated experimental autoimmune encephalomyelitis (EAE) in a preventative disease model in mice.

FIG. 4 shows that KL001 treatment, but not UA, ameliorated EAE in a therapeutic disease model in mice.

FIG. 5 shows that KL001 treatment ameliorated psoriatic symptoms in a preventive imiquimod (IMQ)-induced psoriasis model.

FIG. 6 shows the UA derived chemicals 201702-1 to 201702-4 and 201509-1 to 201509-3 inhibited Th17 differentiation in in vitro cultures.

FIG. 7 shows the structure of ursolic acid, KL001, KL002, KL003, KL004 and KL005.

### DETAILED DESCRIPTION OF THE INVENTION

Th17 cells have recently emerged as a major player in inflammatory and autoimmune diseases via the production of pro-inflammatory cytokines IL-17, IL-17F, IL-21 and IL-22. Th17 cells have been recently discovered as the third effector CD4+ T helper subset. Park, H., et al., A Distinct Lineage of CD4 T Cells Regulates Tissue Inflammation by Producing Interleukin 17, Nat Immunol. 6:1133-1141 (2005); Harrington, L. E., et al., Interleukin 17-Producing CD4+ Effector T Cells Develop via a Lineage Distinct From the T Helper Type 1 and 2 Lineages, Nat Immunol. 6:1123-1132 (2005). Th17 cells produce IL-17, IL-17F and IL-22. Dong, C., TH17 Cells in Development: An Updated View of Their Molecular Identity and Genetic Programming, Nat Rev Immunol. 8:337-348 (2008); Korn, T., et al., IL-17 and Th17 Cells, Annu Rev Immunol. 27:485-517 (2009). Although Th17 cells play important roles in host defense against bacterial and fungal infections, they have been also linked to many immune-related diseases, including psoriasis, rheumatoid arthritis, multiple sclerosis, inflammatory bowel diseases, periodontal diseases and asthma airway inflammatory diseases. Korn, T., et al., IL-17 and Th17 Cells, Annu Rev Immunol. 27:485-517 (2009); Tesmer, L. A., et al., Th17 Cells in Human Disease, Immunol Rev. 223:87-113 (2008). Recently, the anti-IL-17 and anti-IL-17RA antibodies were shown to have good efficacy in treatment of multiple human diseases in phase II or phase III clinical trials. Tse, M. T. IL-17 antibodies gain momentum, Nature reviews. Drug discovery 12, 815-816 (2013); Gaffen, S. L., Jain, R., Garg, A. V. and Cua, D. J. The IL-23-IL-17 immune axis: from mechanisms to therapeutic testing, Nature reviews Immunology 14: 585-600 (2014); Yang, J., Sundrud, M. S., Skepner, J. and Yamagata, T. Targeting Th17 cells in autoimmune diseases, Trends in pharmacological sciences 35: 493-500 (2014).

In Th17 cells, the transcription of IL-17, IL-17F, IL-21 and IL-22 is mediated by Th17-specific transcriptional regulators RORγt and RORα, though the latter plays a less significant role in mice. Ivanov, I. I., et al., The Orphan Nuclear Receptor RORγt Directs the Differentiation Program of Proinflammatory IL-17+ T Helper Cells, Cell 126:1121-1133 (2006); Yang, X. O., et al., T Helper 17 Lineage Differentiation Is Programmed by Orphan Nuclear Receptors RORα and RORγt, Immunity 28:29-39 (2008). Mice deficient in RORγt and those deficient in both RORγt and RORα are defectively in production of IL-17, IL-17F, IL-21 and IL-22, and are resistant to experimental autoimmune encephalomyelitis (EAE), a mouse model for multiple sclerosis. Ivanov, I. I., et al., The Orphan Nuclear Receptor RORt Directs the Differentiation Program of Proinflammatory IL-17+ T Helper Cells, Cell 126:1121-1133 (2006); Yang, X. O., et al., T Helper 17 Lineage Differentiation Is Programmed by Orphan Nuclear Receptors RORα and RORγt, Immunity 28:29-39 (2008). In addition, the differentiation of Th17 cells and the associated cytokine production are directly controlled by RORγt together with a related transcription factor RORα. For example, it has been reported that the expression of IL-17 and development of Th17 cells are directly controlled by retinoid-related orphan receptor RORγt. Ivanov, I. I., et al., The Orphan Nuclear Receptor RORγt Directs the Differentiation Program of Proinflammatory IL-17+ T Helper Cells, Cell 126:1121-1133 (2006); Ivanov, I. I., et al., The Orphan Nuclear Receptor RORγt Directs the Differentiation Program of Proinflammatory IL-17+ T Helper Cells, Cell 126:1121-1133 (2006). Noteworthy, to date, although a number of RORγt inhibitors have been identified to inhibit Th17 differentiation in vitro or in vivo, their effect in clinical application in treatment of Th17-related diseases has not yet been reported. Yang, J., Sundrud, M. S., Skepner, J. and Yamagata, T. Targeting Th17 cells in autoimmune diseases, Trends in pharmacological sciences 35: 493-500 (2014).

Moreover, until the present disclosure, there has been no effective therapy for controlling of excessive TH17 responses and related autoimmune diseases. Now, through the inhibition of RORγt via UA, KL001 and related analogs, it is shown that RORγt inhibitors can be useful to treat autoimmune disease, inflammation, cancer, and immunity disorders related to excellular bacteria, fungus, and viruses, and other diseases and disorders associated with the over expression of: IL-17; IL-17F; IL-21; and IL-21, the production of one or more of the pro-inflammatory cytokines: IL-17; IL-17F; IL-21; and IL-22, and/or generally excessive Th17 cell response. See e.g., Korn, T., et al., IL-17 and Th17 Cells, Annu Rev Immunol. 27:485-517 (2009). More specifically, through the inhibition of RORγt via UA, KL001 and related UA analogs, it is shown that RORγt inhibitors can be useful to treat multiple sclerosis, psoriasis, asthma, inflammatory bowel disease, arthritis, melanoma, rheumatoid arthritis, systemic lupus erythematosus, allograft rejection, ankylosing spondilitis, scleroderma, Type I diabetes, psoriatic arthritis, osteoarthritis, and atopic dermatitis.

IL-21 regulates the differentiation of CD4+ T cells into TH17 cells in an autocrine manner. Expression of IL-21 is induced in T cells by IL-6 via STAT3 and is necessary in the generation of TH17 cells via STAT3-dependent upregulation of RORγt. IL-21 acts in an autocrine fashion in the differentiation of TH17 cells as IFN-γ does for TH1 cells and IL-4 for TH2 cells. Furthermore, the differentiation of TH17 cells can be modulated via the IL-21 signaling pathway. Moreover, any interruption to the pathway upstream of STAT3 will prevent the activation of STAT3, the differentiation of TH17 cells and ultimate expression of IL-21 and other cytokines expressed by the TH17 cells. US Pub. App. No. 2010/0247547 Paragraphs 53, 54, 55, 56, 57 and 58, and FIG. 12, incorporated herein by reference. As such, RORγt inhibitors taught herein suppress specific cytokine signaling pathways such as IL-21 and modulate the differentiation of THI7 cells. Moreover, the RORγt inhibitors may be useful in combination therapies, for example, with STAT3 small molecule inhibitors and IL-21 inhibitors to treat the diseases described herein.

The phrase “therapeutically effective” is intended to qualify the amount of active ingredients used in the treatment of a disease or disorder. This amount will achieve the goal of reducing or eliminating the said disease or disorder. The term “patient” means all mammals including humans. Examples of patients include humans, cows, dogs, cats, goats, sheep, pigs, and rabbits. In one embodiment, the patient is a human.

While it may be possible for the molecules which inhibit RORγt activity to be administered as the raw chemical, it is also possible to present them as a pharmaceutical formulation. Accordingly, the pharmaceutical formulation may include the molecule or a pharmaceutically acceptable a salt, ester, prodrug or solvate thereof, where appropriate, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences.

The formulations of use molecules include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods may include the step of bringing into association the molecule or a pharmaceutically acceptable salt, ester, prodrug or solvate thereof (“active ingredient”) with the carrier which constitutes one or more accessory ingredients.

In certain instances, as noted above, it may be appropriate to administer a RORγt inhibitor (or a pharmaceutically acceptable salt, ester, or prodrug thereof) in combination with another therapeutic agent such as a STAT3 inhibitor or IL-21 inhibitor. Or, by way of example only, the therapeutic effectiveness of the inhibitors provided herein may be enhanced by administration of an adjuvant (i.e., by itself the adjuvant may only have minimal therapeutic benefit, but in combination with another therapeutic agent such as a STAT3 inhibitor, the overall therapeutic benefit to the patient is enhanced). Or, by way of example only, the benefit of experienced by a patient may be increased by administering one molecule as described herein with another therapeutic agent (which also includes a therapeutic regimen) that also has therapeutic benefit.

Multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses.

In previous studies, ursolic acid (UA), a natural carboxylic acid ubiquitously present in plants, has been identified as a strong and selective inhibitor for RORγt function and TH17 differentiation. UA inhibited IL-17 production not only in developing Th17 cells but also in mature Th17 cells. Xu, T., et al. Ursolic acid suppresses interleukin-17 (IL-17) production by selectively antagonizing the function of RORgamma t protein. The Journal of biological chemistry 286: 22707-22710 (2011). In addition, UA treatment ameliorated experimental autoimmune encephalomyelitis (EAE) in a preventative disease model. Xu, T., et al. Ursolic acid suppresses interleukin-17 (IL-17) production by selectively antagonizing the function of RORgamma t protein. The Journal of biological chemistry 286: 22707-22710 (2011). Based on these studies, a series of UA derived chemicals was generated, among which it was found that KL001, though inhibited Th17 differentiation in vitro at a comparable level to UA, behaved better than UA in treatment of EAE disease in both preventative and therapeutic disease models. In addition, it was found that KL001 can significantly reduce the disease symptoms in a mouse model of psoriasis. As such, it is shown that KL001 and other UA derived chemicals, through releasing UA as the major active ingredient and via targeting RORγt, are viable drug products for developing treatments against Th17-mediated inflammatory diseases and cancer.

As described herein, UA, KL001 and other UA derived chemicals shown in Examples below can selectively and effectively blocked the function of RORγt and IL-17 expression in both differentiated and developing Th17 cells and can be used in diseases associated with IL-17 expression and differentiation of Th17 cells. UA is a relatively non-toxic natural small molecule having a long history in herbal medicine practice. UA can be useful for the treatment of liver diseases, skin cancer and non lymphatic leukemia. In addition, treatment with UA has been shown to ameliorate a mouse model of human multiple sclerosis. Liu, J. Pharmacology of Oleanolic Acid and Ursolic Acid, J Ethnopharmacol. 49:57-68 (1995). Since KL001 and other UA derived chemicals can release UA as the major active ingredient under in vitro and in vivo settings, these UA analogs can be also used in treatment of these listed UA-treatable diseases.

As noted above, to date, the role of RORγt inhibitors in clinical application in treating Th17-related diseases has not been reported. Yang, J., Sundrud, M. S., Skepner, J. and Yamagata, T. Targeting Th17 cells in autoimmune diseases, Trends in pharmacological sciences 35: 493-500 (2014). Moreover, until the present disclosure, there has been no effective therapy for controlling of excessive TH17 responses and related autoimmune diseases. Now, through the inhibition of RORγt, it is postulated that RORγt inhibitors can treat multiple sclerosis, autoimmune disease, asthma, inflammatory bowel disease, inflammation, cancer, multiple sclerosis, arthritis, rheumatoid arthritis, asthma, systemic lupus erythematosus, allograft rejection, psoriasis, ankylosing spondilitis, scleroderma, Type I diabetes, psoriatic arthritis,osteoarthritis, and atopic dermatitis, and immunity disorders related to excellular bacteria, fungus, and viruses, and any disease associated with the over-expression of IL-17, IL-17F, IL-21 and IL-21, the production of one or more of the pro-inflammatory cytokines IL-17, IL-17F, IL-21 and IL-22, and/or generally excessive Th17 cell response.

It has been demonstrated that UA, KL001 and other UA analogs inhibit TH17 differentiation under in vitro and in vivo animal disease models. By using both preventative and therapeutic EAE models, as well as IMQ-induced psoriasis model, it was demonstrated that RORγt inhibitors can be used to cure TH17-related diseases. More importantly, KL001 releases UA as the major active ingredient in these animal disease models, while UA is a natural product present in many medical herbs and human diet, and has already been demonstrated to be relatively non-toxic (LD50>600 mg/kg body weight in rodents for intraperitoneal injection. Gautam, R. & Jachak, S. et al., Recent Developments in Anti-Inflammatory Natural Products, Med. Res. Rev. 29:767-820 (2009). Indeed, it has been already recommended for skin cancer therapy in Japan. Muto, Y., et al., Present Status of Research on Cancer Chemoprevention in Japan, Japanese J. Clin. Oncology 20:219-224 (1990). The selective effect of KL001 or other UA analogs on Th17 cells, together with the low toxicity and long-term medical practice of their major active ingredient UA, provides KL001 and other UA analogs a great advantage over other non-UA related RORγt inhibitors in clinical applications for developing treatment of Th17 related-autoimmune diseases.

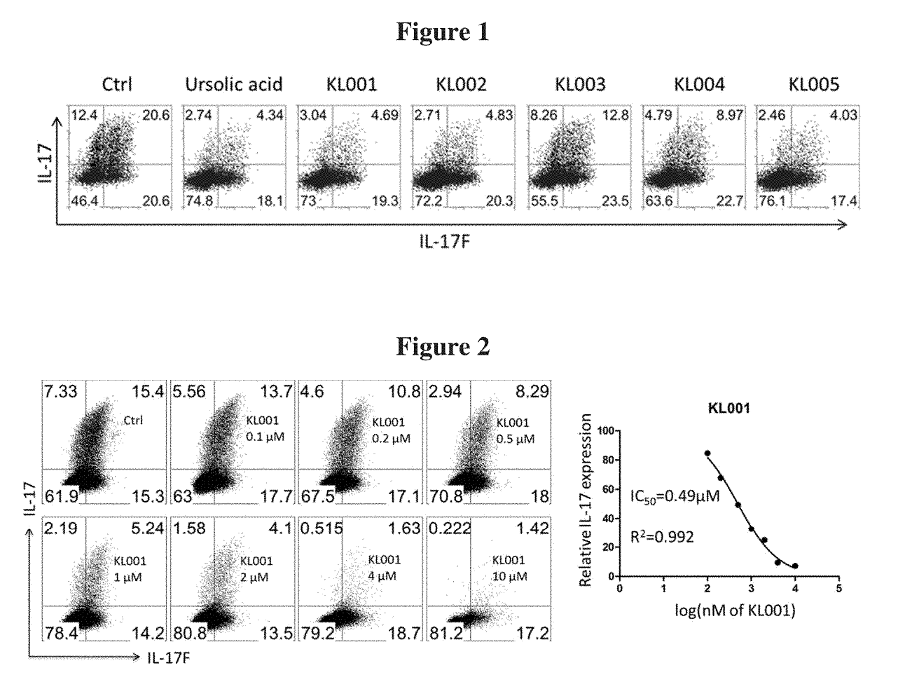

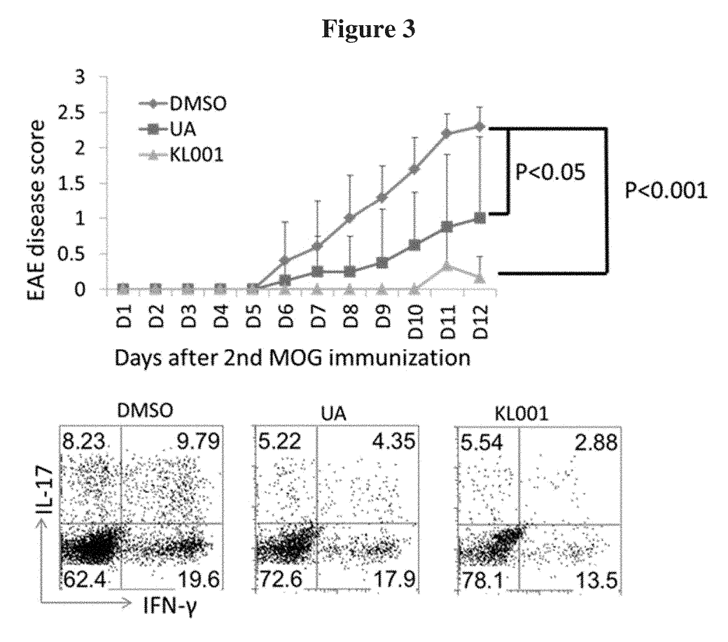

Ursolic acid (UA) is a relatively non-toxic natural pentacyclictriterpenoid carboxylic acid present in numerous plants, medical herbs and even human diet. Gautam, R. & Jachak, et al., Recent Developments in Anti-Inflammatory Natural Products, Med. Res. Rev. 29:767-820 (2009). UA has been shown to have different pharmacological activities, including anti-tumor and anti-inflammation effects. Liu, J. Pharmacology of Oleanolic Acid and Ursolic Acid, J Ethnopharmacol. 49:57-68 (1995); Ikeda, Y., et al., Ursolic Acid: An Anti- and Pro-Inflammatory Triterpenoid, Mol Nutr Food Res. 52:26-42 (2008). While useful in a wide variety and number of different types of treatments and disease, for the first time, it was demonstrated that UA is also a specific and selective inhibitor for RORγt and TH17 cells. To search for more effective RORγt and TH17 inhibitors, a series of UA analogs was developed and examined their role in inhibiting TH17 cells in an in vitro differentiation system. As taught below, it was found that several of them including KL001, KL002 and KL005 showed comparable efficacy with UA in inhibiting TH17 differentiation (FIG. 1). After a further investigation, it was demonstrated that KL001 was superior to UA in ameliorating EAE diseases in both preventative and therapeutic disease models in mice (FIGS. 2 and 3), possibly due to better bio-availability in in vivo exposures.

Taken together, these data strongly suggest UA, KL001 and UA related analogs disclosed immediately below in the examples are valuable drug candidates for treatment of Th17-related autoimmune diseases, as well as for the diseases targeted by UA in clinical application, including liver diseases and skin cancer. Liu, J. Pharmacology of Oleanolic Acid and Ursolic Acid, J Ethnopharmacol. 49:57-68 (1995).

In one embodiment, the present invention provides a compound for modulating ROR-gamma. The compound has the structure of:

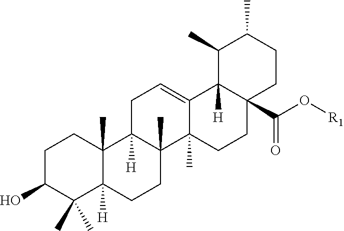

or a physiologically acceptable salt or hydrate, or solvate thereof, wherein R₁ is selected from the group consisting of:

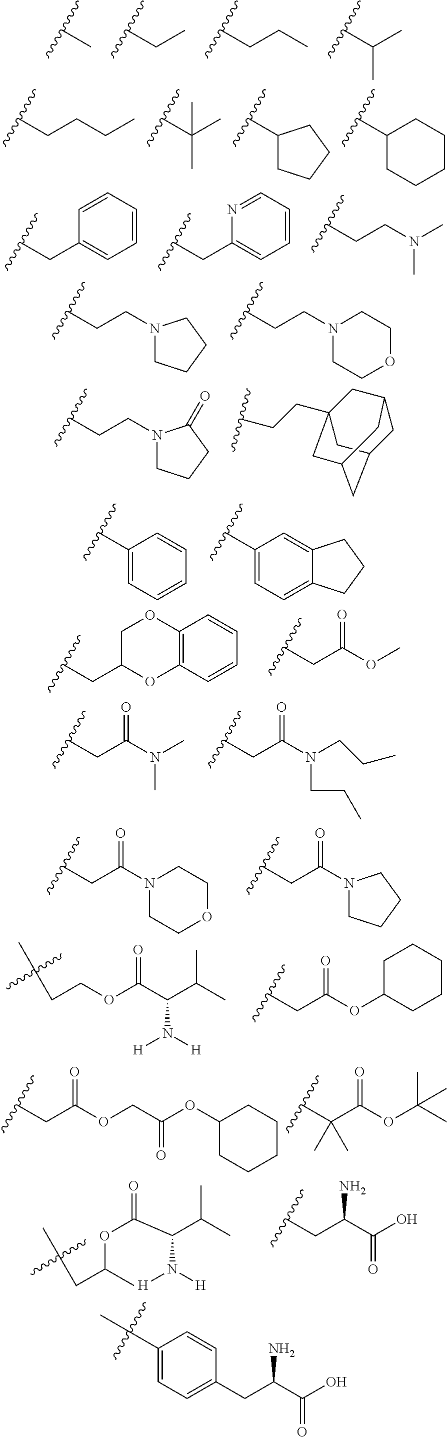

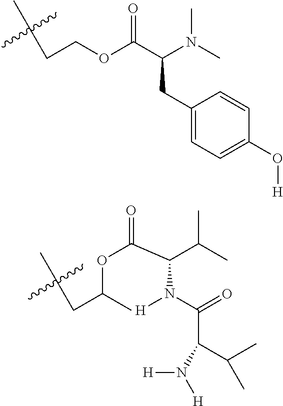 [SMILES: *CCOC(=O)[C@H](Cc1ccc(O)cc1)N(C)C ; *CC(C)OC(=O)[C@@H](NC(=O)[C@@H](N)C(C)C)C(C)C]

or R₁ is selected from the group consisting of:

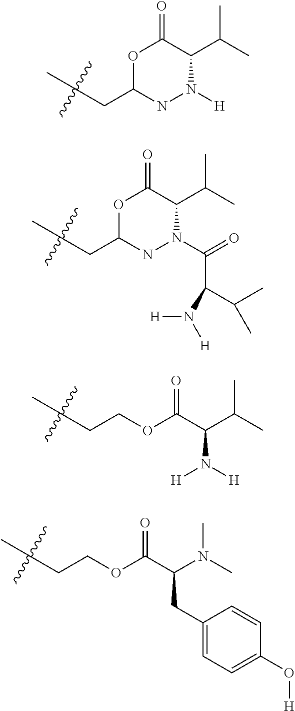

In another embodiment, the compound has the structure of:

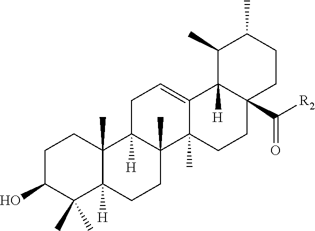

wherein R₂ is selected from the group consisting of:

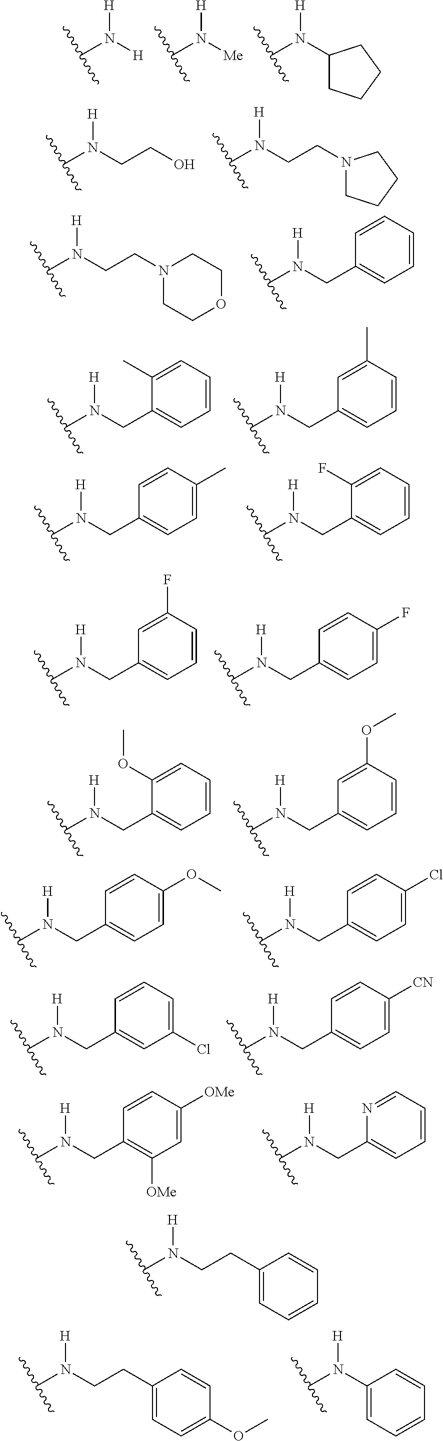

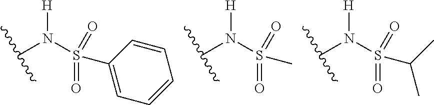

or R₂ is selected from the group consisting of:

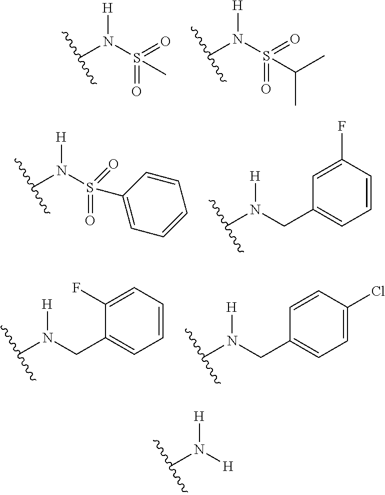

In another embodiment, the compound has the structure of:

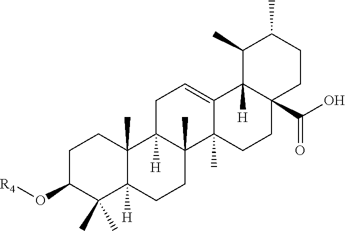

wherein R₄ is selected from the group consisting of:

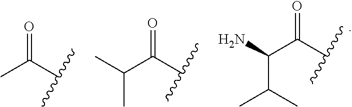

The present invention also provides a composition comprising any one of the compounds described above, wherein the composition comprises at least one pharmaceutically acceptable carrier or diluent.

In one embodiment, the present invention provides a composition comprising any one of the compounds described above, in a form of a tablet, a capsule, an injectable composition, an ingestible composition, a nasal spray, an aerosol, or a suppository.

In one embodiment, the present invention provides a method of inhibiting differentiation of a population of T cells or Th17 cells by contacting the T cells or Th17 cells with a composition containing a compound having the structure of:

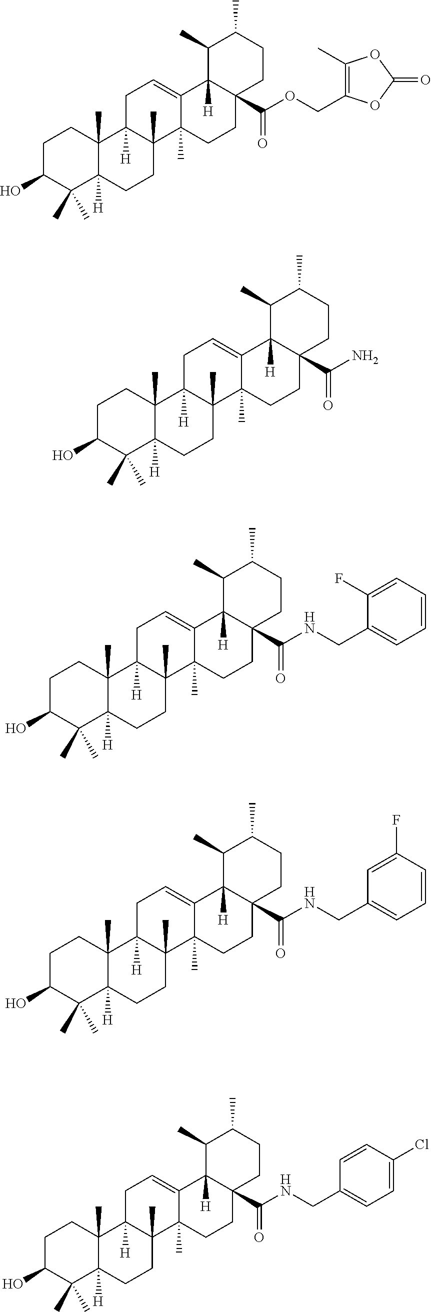

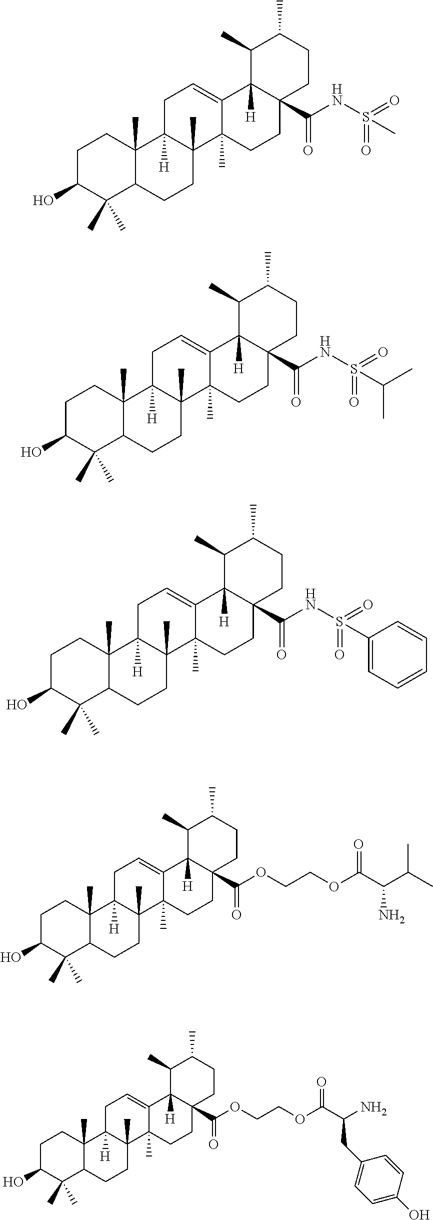

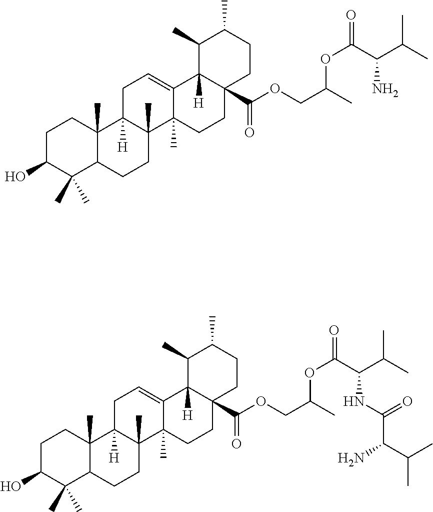

or a physiologically acceptable salt, hydrate, or solvate thereof.

In another embodiment, the present invention provides a method of treating a disease caused by Th17 cell activities in a subject in need of such treatment, said method comprises administering to said subject a composition comprising a therapeutically effective amount of a compound having the structure of:

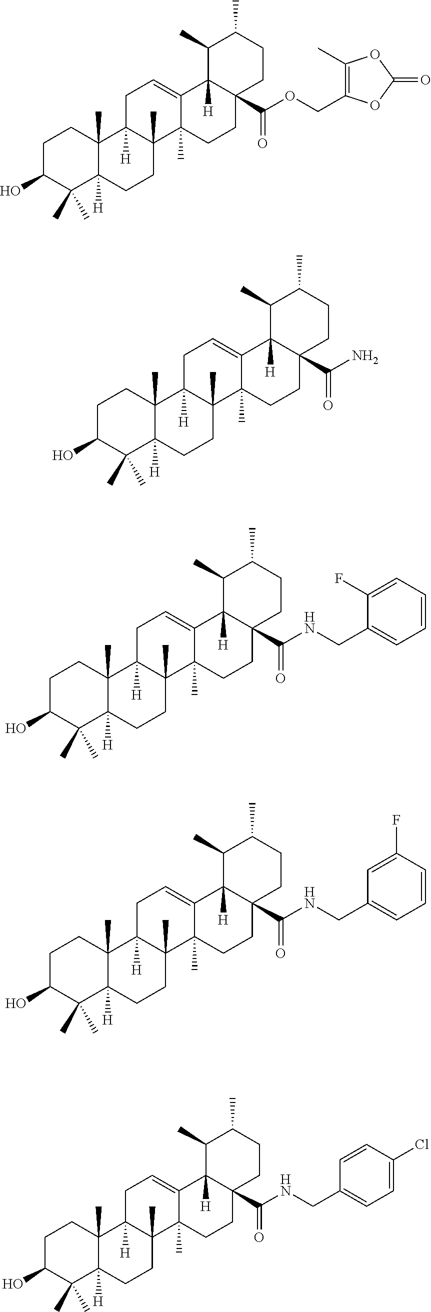

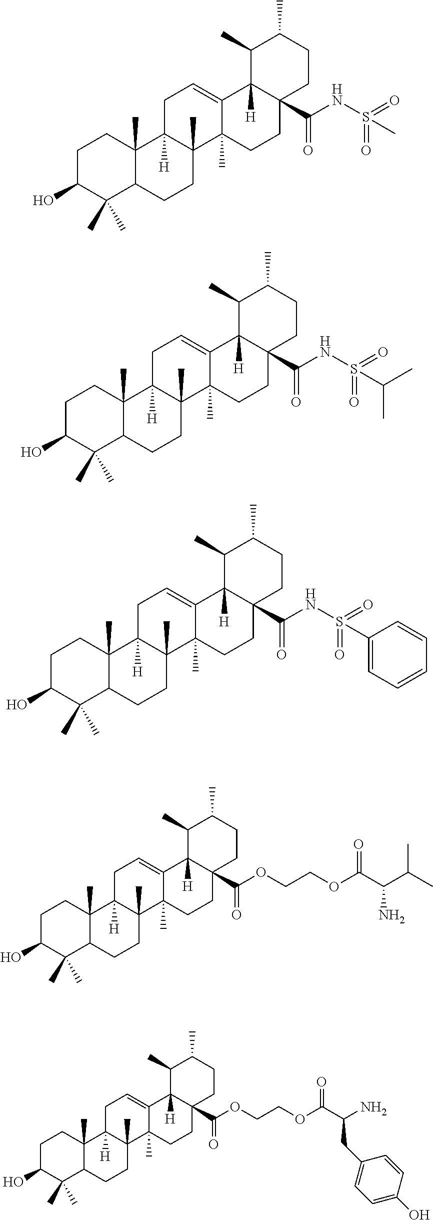

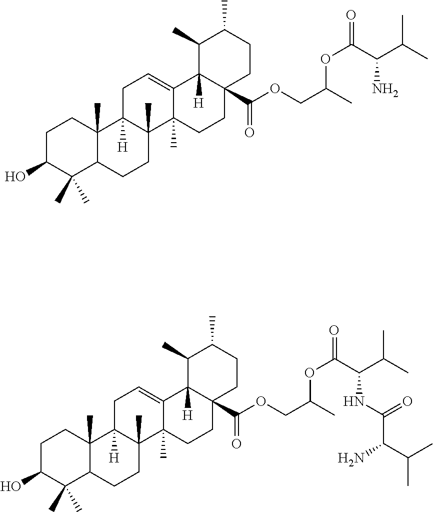

or a physiologically acceptable salt, hydrate, or solvate thereof.

In one embodiment, the present invention uses the aforementioned method to treat an inflammatory disease, autoimmune disease, or a disease related to excessive function of Th17 cells.

In one embodiment, the present invention uses the described method to treat autoimmune encephalomyelitis, collagen-induced arthritis, multiple sclerosis, rheumatoid arthritis, psoriasis, Crohn's disease, asthma, inflammatory bowel disease, arthritis, melanoma, systemic lupus erythematosus, allograft rejection, ankylosing spondilitis, scleroderma, Type I diabetes, psoriatic arthritis, osteoarthritis, or atopic dermatitis.

In one embodiment, the composition of the present invention is in the form of a tablet, capsule, injectable composition, ingestible composition, nasal spray, aerosol or a suppository.

In one embodiment, the composition of the present invention is administered in a route selected from a group consisting of mucosal, oral, nasal, topical, transdermal, intradermal, parenteral, intraperitoneal, intramuscular, intravenous, subcutaneous, rectal, intraarticular, intramedullary, intraocular, buccal, and sublingual application.

In one embodiment of the above method, the compound is administered in a daily dosage of about 100 to 500 mg/kg body weight of the subject. In another embodiment, the compound is administered in a daily dosage of about 500 mg/kg body weight. In another embodiment, the compound is administered in a daily dosage of about 50-250 mg/kg body weight.

In one embodiment, the above method comprises administering a second therapeutic agent or treatment for the aforementioned diseases.

In one embodiment of the above method, the method further comprises administering to the subject a second therapeutic agent or treatment for said disease. In one embodiment, the second therapeutic agent is a STAT3 inhibitor, an IL-21 inhibitor, or an IL-22 inhibitor.

### Methods of Preparation

The compounds of the present invention may be prepared by methods as those illustrated in the following scheme I to II. Starting materials are commercially available or prepared by the methods as those illustrated in the following scheme III to V. Additional methods for making selected compounds of the present invention are provided below. Solvents, temperatures, pressures and other reaction conditions may readily be selected by one of ordinary skill in the art.

### EXAMPLE 1

### KL001

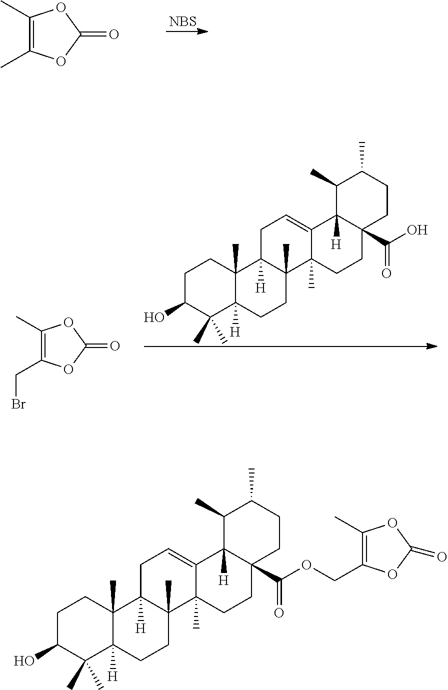

Step 1: To a solution of 16 grams 4,5-dimethyl-1,3-dioxol-2-one in CCl4 (250 mL) was added NBS (23.4 g, 131.5 mmol) and AIBN (1.3 g, 7.88 mmol), then the reaction mixture was heated to 80° C. and stirred for 2 hrs. Monitored the starting material gone by TLC. The mixture was filtered and the filtrate was washed with water and brine, the organic phase was dried over Na₂SO₄ and concentrated to give crude product as a yellow oil which was used directly next step (25.3 g, yield 93%).

Step 2: K₂CO₃ (300 mg, 2 mmol) was added to a solution of 4-(bromomethyl)-5-methyl-1,3-dioxol-2-one (390 mg, 2 mmol) and Ursolic acid (460 mg, 1 mmol) in 20 mL acetone, then the reaction mixture was stirred at 55° C. for 24 hours. The mixture was concentrated to removed acetone, 100 mL water was added, the solid was filtered and washed with water, dried to give product. (385 mg, yield: 68%) as a white solid. ¹HNMR (CDCl3, 300 MHz): δ 5.25˜5.26 (t, 1H, J=4.8 Hz), 4.67˜4.87 (q, 2H), 3.18-3.24 (m, 1H), 2.19˜2.24 (m, 1H), 2.15˜2.16 (d, 3H), 1.18˜1.98 (m, 23H), 1.08 (s, 3H), 0.99 (s, 3H), 0.86˜0.88 (d, 3H), 0.85 (s, 3H), 0.77˜0.79 (d, 3H, J=8.4 Hz), 0.79 (s, 3H), 0.65 (s, 3H). ESI-MS m/z 569.4 [M+H].

### EXAMPLE 2

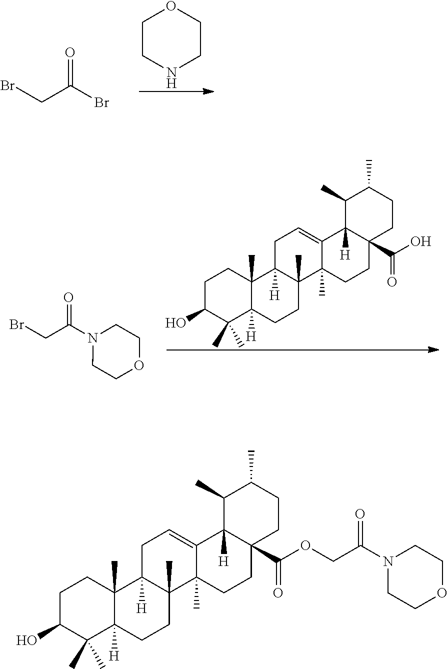

Step 1: To a solution of 2-bromoacetyl bromide (2.3 g, 11.4 mmol) in 50 mL DCM was added morpholine (1.22 g, 14 mmol) and Et₃N (1.9 mL, 13.7 mmol). The reaction was stirred at room temperature for 1 hr. The mixture was washed with water, brine, dried and concentrated to give crude product used directly.

Step 2: K₂CO₃ (300 mg, 2 mmol) was added to a solution of 2-bromo-1-morpholinoethan-1-one (412 mg, 2 mmol) and Ursolic acid (460 mg, 1 mmol) in 20 mL acetone, then the reaction mixture was stirred at room temperature for overnight. The mixture was concentrated to removed acetone, 100 mL water was added, the solid was filtered and washed with water, dried to give product. (408 mg, yield: 70%) as a white solid. ¹HNMR (CDCl3, 300 MHz): δ 5.16˜5.18 (t, 1H, J=4.8 Hz), 4.51˜4.62 (q, 2H), 3.5-3.6 (m, 8H), 3.11-3.18 (m, 1H), 2.90 (s, 3H), 2.88 (s, 3H), 2.13˜2.17 (m, 1H), 1.18˜1.98 (m, 23H), 1.01 (s, 3H), 0.92 (s, 3H), 0.86˜0.88 (d, 3H), 0.85 (s, 3H), 0.7˜0.79 (d, 3H, J=8.4 Hz). ESI-MS m/z 584.4 [M+H].

### EXAMPLE 3

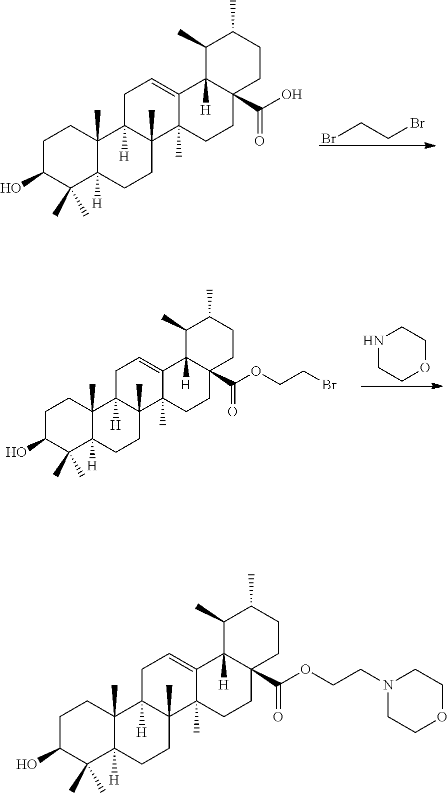

Step 1: A mixture of Ursolic acid (1 g, 2.19 mmol), 1,2-dibromoethane (1 mL, 11.5 mmol) and K2CO3 (800 mg, 5.8 mmol) in 80 mL DMF was stirred at 50° C. in the seal tube for 8 hrs. The reaction mixture was concentrated, 400 mL water was added, the solid product was filtered and dried used directly.

Step 2: K₂CO₃ (300 mg, 2 mmol) was added to step 1 product solution (560 mg, 1 mmol) and morpholine (175 mg, 2 mmol) in 100 mL acetone, then the reaction mixture was heated to 50° C. for 48 hrs. The mixture was filtered to remove the solid and the filtrate was concentrated and purified by flash column (Hexane/EA=5/1) to afford white solid 353 mg (62% yield). ¹HNMR (CDCl3, 300 MHz): δ 5.15˜5.17 (t, 1H, J=4.8 Hz), 4.04˜4.08 (t, 2H, J=8.0 Hz), 3.62˜3.65 (t, 2H, J=6.0 Hz), 3.12-3.17 (m, 1H), 2.52˜2.56 (t, 2H, J=8.0 Hz), 2.42˜2.45 (t, 2H, J=6.0 Hz), 2.12˜2.16 (m, 1H), 1.19˜1.97 (m, 23H), 1.00 (s, 3H), 0.93 (s, 3H), 0.86˜0.88 (d, 3H), 0.85 (s, 3H), 0.78˜0.80 (d, 3H, J=8.4 Hz), 0.72 (s, 3H), 0.69 (s, 3H). ESI-MS m/z 570.3 [M+H].

### EXAMPLE 4

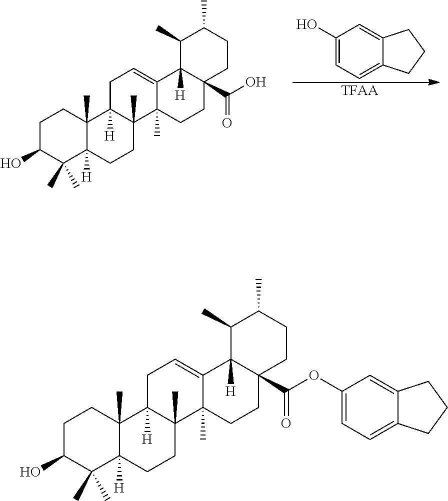

Step 1: A mixture of Ursolic acid (46 mg, 0.11 mmol) and TFAA(0.056 mL) in 10 mL toluene was stirred at room temperature for 1 hr, then 2,3-dihydro-1H-inden-5-ol (17 mg, 0.12 mmol) was added and the reaction was refluxed for 24 hrs. The reaction mixture was concentrated, the solid crude product purified by Pre-TLC (PE/EA=1/1) to afford product as a white solid (47 mg, 75% yield). ¹HNMR (CDCl3, 300 MHz): δ 7.14˜7.16 (d, 1H, J=10.4 Hz), 6.84 (s, 1H), 6.71˜6.74 (dd, 1H, J=10.4 Hz), 5.30˜5.31(t, 1H, J=4.8 Hz), 3.20-3.25 (m, 1H), 2.84˜2.91 (q, 4H, J=9.6 Hz), 2.33˜2.37 (m, 1H), 1.20˜2.15 (m, 25H), 1.13 (s, 3H), 1.00 (s, 3H), 0.97˜0.99 (d, 3H), 0.94(s, 3H), 0.88˜0.90 (d, 3H, J=8.4 Hz), 0.90 (s, 3H), 0.79 (s, 3H). ESI-MS m/z 573.3 [M+H].

### EXAMPLE 5

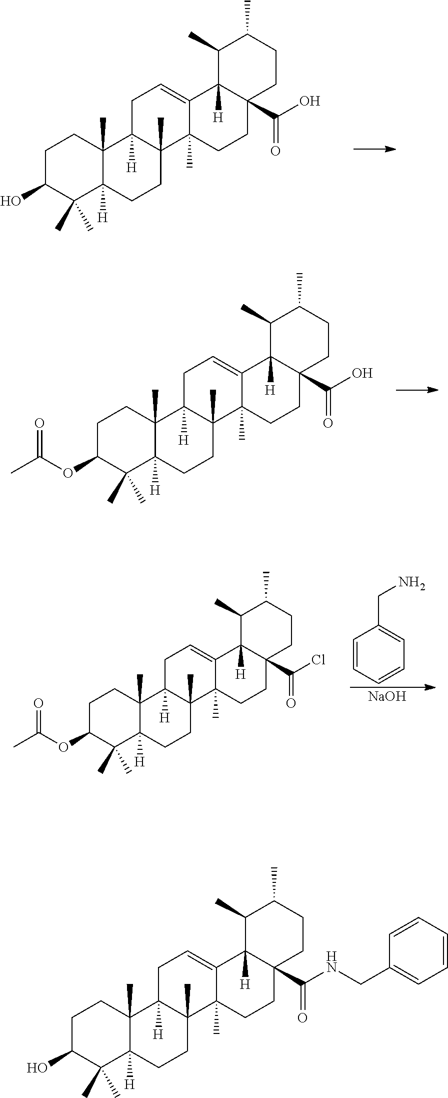

Step 1: A mixture of ursolic acid (500 mg, 1.09 mmol), Ac₂O (0.16 mL) and DMAP (25 mg) in 2.5 mL pyridine was stirred at room temperature for overnight. The reaction mixture was concentrated, 10 mL water was added, and adjust pH to 3˜4 with 0.1N HCl. The solid product was filtered and dried without further purification.

Step 2: C₂O₂Cl₂ (0.2 mL, 2.1 mmol) was added to a solution of step 1 product (300 mg, 0.6 mmol) in 30 mL DCM, the reaction was stirred at room temperature for overnight. The reaction solution was used directly in next step.

Step 3: phenylmethanamine (1 eq) was added to a solution of step 2. The reaction was stirred at room temperature for overnight. The reaction solution was adjust pH=3˜4 with 1N HCl, and removed DCM in vacuum. The residue was dissolved in THF, added moderate amount MeOH, then drop-wise added 15% aq. NaOH. The reaction mixture was stirred at room temperature for overnight. Moderate amount water was added to reaction, adjust pH=3˜4 with 1N HCl, removed THF and MeOH. The precipitate solid was filtered, washed with a small amount MeOH to get product as white solid. ¹HNMR (CDCl3, 300 MHz): δ 7.26˜7.33 (m, 5H), 6.14˜6.18 (m, 1H), 5.21˜5.23 (m, 1H), 4.12˜4.61 (m, 2H), 3.20-3.24 (m, 1H), 1.28˜2.06 (m, 24H), 1.10 (s, 3H), 1.00 (s, 3H), 0.96 (s, 3H), 0.90(s, 3H), 0.85˜0.87 (d, 3H, J=8.8 Hz), 0.80 (s, 3H), 0.72 (s, 3H). ESI-MS m/z 546.3 [M+H].

### EXAMPLE 6

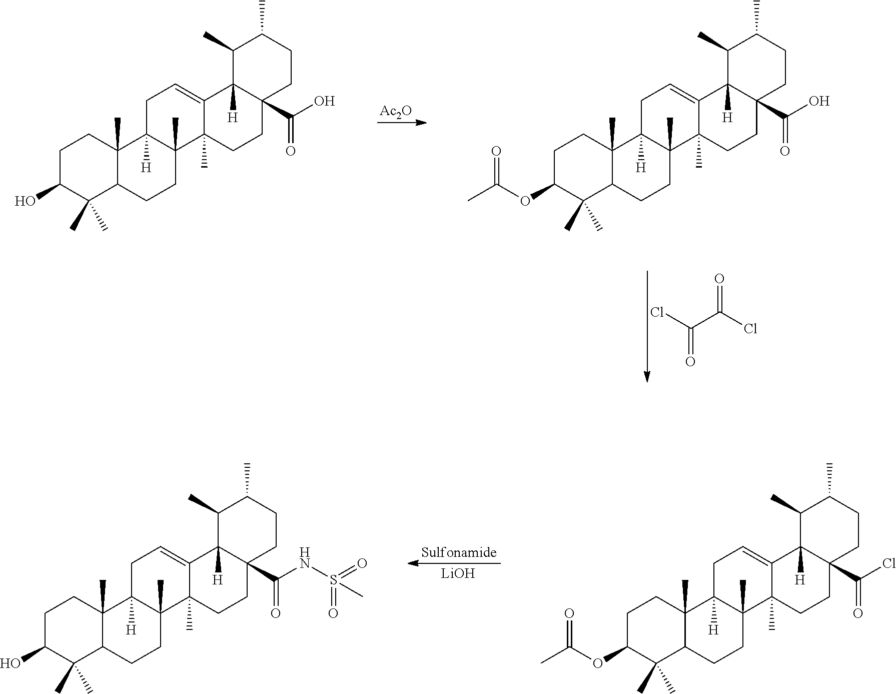

Step 1: A mixture of Ursolic acid (500 mg, 1.09 mmol), Ac₂O (0.16 mL) and DMAP (25 mg) in 2.5 mL pyridine was stirred at room temperature for overnight. The reaction mixture was concentrated, 10 mL water was added, and adjust pH=3˜4 with 0.1N HCl, the solid product was filtered and dried to yield target compound as white solid. Yield: 580 mg.

Step 2: DMF (10 ul), oxalyl chloride (0.46 mL, 5.45 mmol) was added to a solution of step 1 product (580 mg, 1.09 mmol) in 10 mL DCM, the reaction was stirred at room temperature overnight. The crude product was concentrated under vacuum used directly in the next step.

Step 3: Sulfonamide (1 eq) was added to a solution of step 2 product in DCM (10 ml), TEA (5 eq) and DMAP (1.1 eq) were added to this solution. The reaction was stirred at 40° C. overnight, then washed with 10% Citric acid aqueous solution (3×10 mL). The organic phase was dried with Na₂SO₄ and concentrated under vacuum. The residue dissolved in MeOH, adjust pH˜12 with 15% aq. LiOH, then the reaction mixture was stirred at room temperature overnight. Moderate water was added to reaction, adjust pH=3˜4 with 1N HCl. After removing MeOH, precipitate solid was filtered, washed with a small amount MeOH get product. (1S,2R,4aS,6aS,6bR,10S,12aR,12bR,14bS)-10-hydroxy-1,2,6a,6b,9,9,12a-heptamethyl-N-(methylsulfonyl)-1,3,4,5,6,6a,6b,7,8,8a,9,10,11,12,12a,12b,13,14b-octadecahydropicene-4a(2H)-carboxamide is a white solid. ESI-MS m/z 534.2 [M+H], ¹HNMR (CDCl3): δ 8.19 (s, 1H), δ 5.42˜5.44 (t, 1H, J=4.8 Hz), 3.26 (s, 3H), 3.19-3.25 (m, 1H), 1.25˜2.11 (m, 25H), 1.12 (s, 3H), 1.00 (s, 3H), 0.97 (s, 3H), 0.94 (s, 3H), 0.87˜0.89 (d, 3H, J=8.4 Hz), 0.87 (s, 3H), 0.79 (s, 3H).

| No. | Structure | NMR/MS |
|---|---|---|
| | 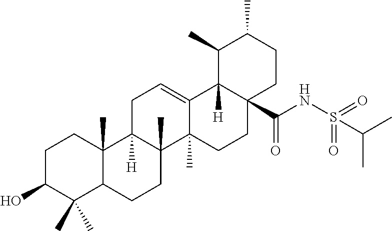 | ¹H NMR (300 MHz, CDCl₃) δ 5.15~5.18 (t, 1H, J = 4.8Hz), 3.53 (s, 3H), 3.11-3.16 (m, 1H), 2.13~2.17 (m, 1H), 1.18~1.98 (m, 23H), 1.01 (s, 3H), 0.92 (s, 3H), 0.86~0.88 (d, 3H), 0.85 (s, 3H), 0.77~0.79 (d, 3H, J = 8.4 Hz), 0.71 (s, 3H), 0.67 (s, 3H). ESI-MS m/z 562.3 [M + H]. |
| | 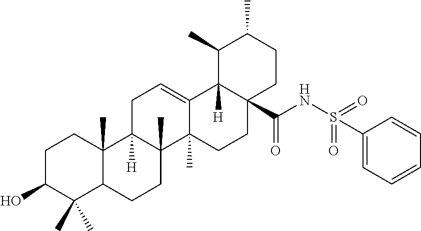 | ¹H NMR (CDCl3): δ 8.28 (s, 1H), 8.03~8.06 (m, 2H), 7.48~7.64 (m, 3H), 5.37~5.40 (t, 1H, J = 4.8Hz), 3.17-3.23 (m, 1H), 1.16~2.20 (m, 24H), 1.06 (s, 3H), 0.98 (s, 3H), 0.94 (d, 3H), 0.90 (s, 3H), 0.84~0.86 (d, 3H, J = 8.4Hz), 0.78 (s, 3H), 0.43 (s, 3H). ESI-MS m/z 596.3 [M + H]. |

### EXAMPLE 7

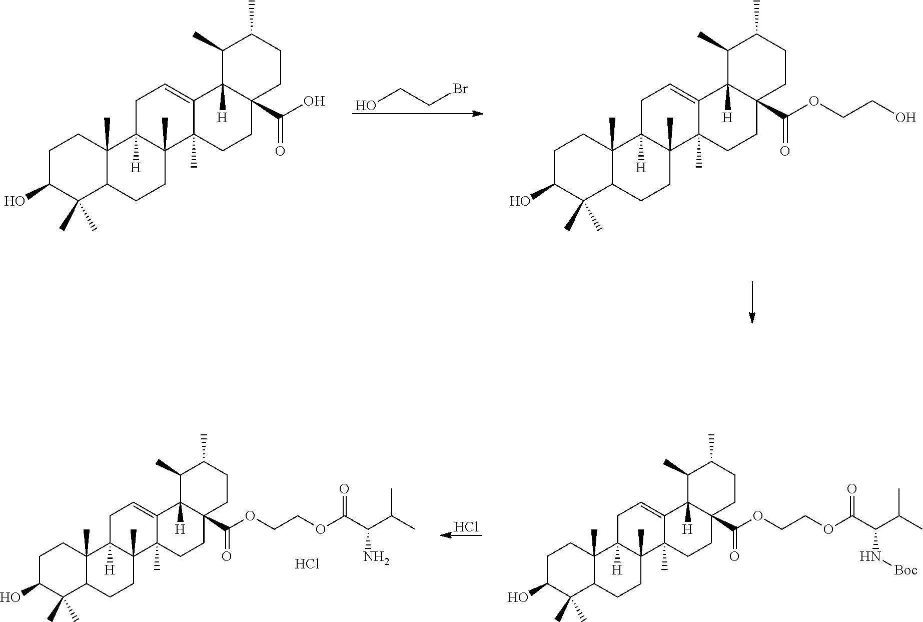

Step 1: To a solution of Ursolic acid (1 g, 2.18 mmol) in DMF (10 mL), 2-bromo ethanol (817 mg, 6.54 mmol) and potassium carbonate (904 mg, 6.54 mmol) were added and the mixture was stirred at room temperature for overnight. Water (50 mL) was added into the filtrate and the obtained mixture was stirred for additional 0.5 h. The precipitate was collected by filtration and washed with water to give the title compound as a white solid 1.1 g.

Step 2: To a solution of step 1 product (400 mg, 0.8 mmol, 1 equiv) in dry CH2Cl2 (20 mL), Boc-L-valine (1.5 equiv), dicyclohexylcarbodiimide (DCC) (1.2 equiv), and 4-dimethyl-aminopyridine (DMAP) (0.5 equiv) were added and the mixture was stirred at room temperature for overnight. After filtration, the filtrate was evaporated to give a crude product which was purified by column chromatography (PE/EtOAc=10:1-8:1) to yield Boc-amino acid ethyl esters as a white solid (400 mg, 71.4%).

Step 3: Dry hydrogen chloride gas was bubbled into a solution of step 2 product (88 mg, 0.125 mmol) in dry ether (3 mL) overnight. The precipitate was collected by filtration and washed with dry ether to give the final product, a white solid (50 mg, 62.8%). ESI-MS m/z 600.3 [M+H], ¹HNMR (CDCl3): δ 8.92 (s, 3H), 5.24 (s, 1H), 3.94-4.48 (m, 5H), 3.18˜3.25 (m, 1H), 1.27˜2.23 (m, 25H), 1.18 (s, 6H), 1.09 (s, 3H), 1.00 (s, 3H), 0.96 (s, 3H), 0.92 (s, 3H), 0.85˜0.87 (d, 3H, J=8.4 Hz), 0.79 (s, 3H), 0.74 (s, 3H).

### EXAMPLE 8

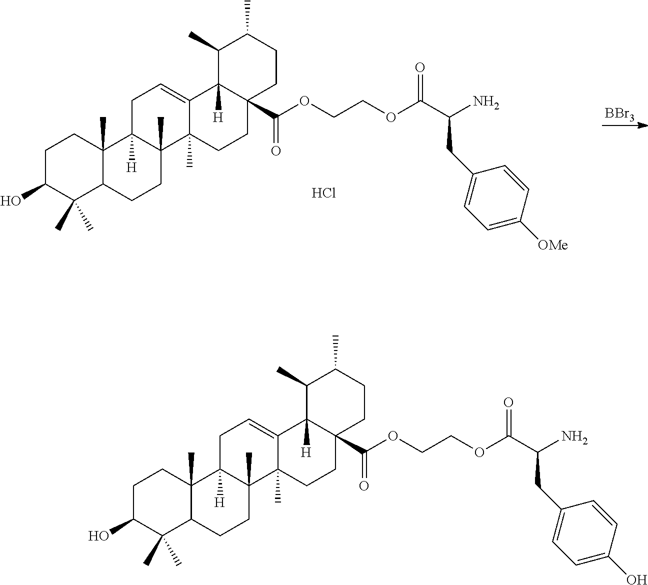

Step 1: To a solution of starting material (197 mg, 0.25 mmol, followed example 7 to make) in dry CH2Cl2 (10 mL), BBr3 (126 ul, 4N, 5 mmol) were drop wise added at −78° C. under Aragon gas and the mixture was stirred at room temperature for overnight. Saturation NaHCO3 (10 ml) was then added drop wise at −20° C. and stirred 0.5 h. The organic phase was dried with anhydrous Na2SO4, and filtrated. Concentrated under vacuum and purified by Pre-TLC get product as white solid. ESI-MS m/z 664.3 [M+H], ¹HNMR (CDCl3): δ 8.92 (s, 3H), 5.24 (s, 1H), 3.94-4.48 (m, 5H), 3.18˜3.25 (m, 1H), 1.27˜2.23 (m, 25H), 1.18 (s, 6H), 1.09 (s, 3H), 1.00 (s, 3H), 0.96 (s, 3H), 0.92 (s, 3H), 0.85˜0.87 (d, 3H, J=8.4 Hz), 0.79 (s, 3H), 0.74 (s, 3H).

### EXAMPLE 9

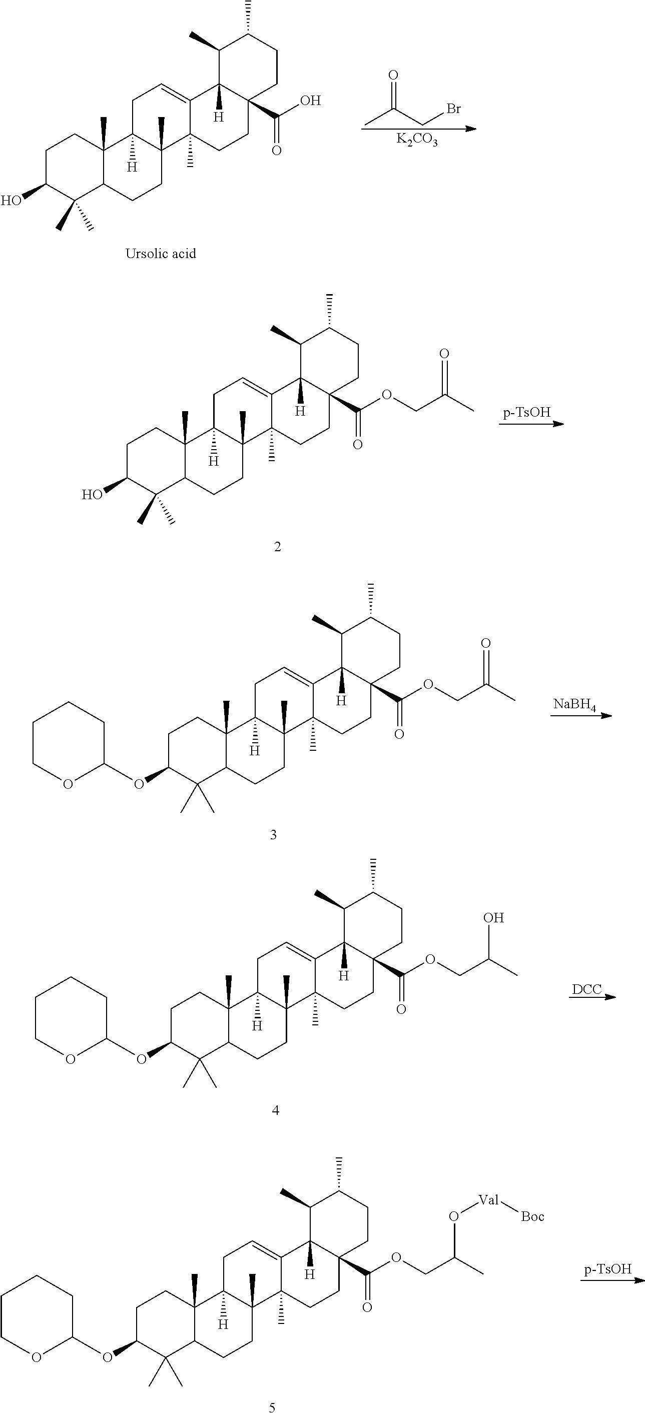

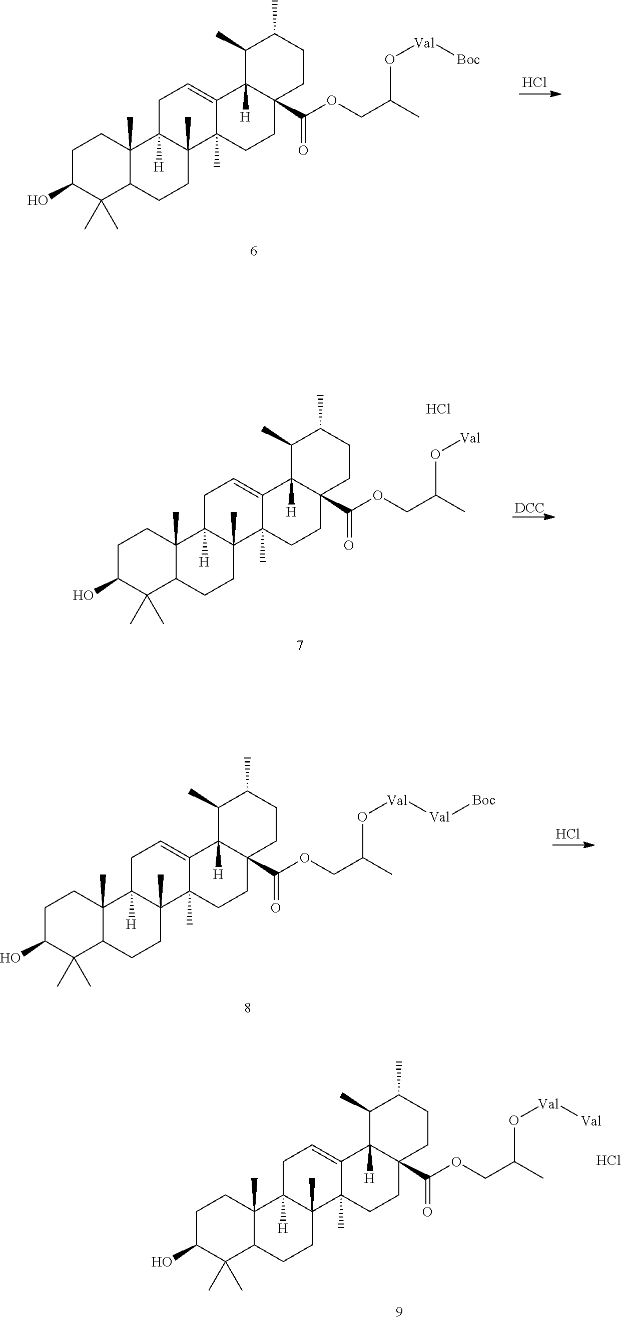

Step 1: To a solution of Ursolic acid (3 g, 6.56 mmol) in DMF (20 mL), 1-bromopropan-2-one (1 mg, 7.3 mmol) and potassium carbonate (2.7 mg, 19.68 mmol) were added and the mixture was stirred at room temperature for overnight. Water (100 mL) was added into the filtrate and the obtained mixture was stirred for additional 0.5 h. The precipitate was collected by filtration and washed with water to give the title compound 2 as a white solid (3.3 g, 98%).

Step 2: To a solution of compound 2 (3.3 g, 6.43 mmol) and pyridinium p-toluenesulfonate (0.017 g, 0.069 mmol) in CH₂Cl₂ (40 mL) a solution of 3,4-dihydro-2H-pyran (2.63 g, 31.25 mmol) in CH₂Cl₂ (20 mL) was added drop wise. The solution was stirred for 3 hours at room temperature, washed by saturated NaHCO₃ aqueous solution (40 mL×3) and saturated NaCl aqueous solution (40 mL×3) respectively, dried with anhydrous Na2SO4, and filtrated. Concentrated under vacuum to get compound 3 used directly in next step (4.6 g, yellow oil).

Step 3: A solution of compound 3 (4.6 g, 6.43 mmol) in methanol (150 mL) was cooled to 0° C., and then NaBH₄ (2.4 g, 64.3 mmol) was added. The mixture was stirred for overnight at room temperature, and then concentrated to remove methanol. The residue was dissolved in EtOAc (50 mL) and washed with water (40 mL×3) and saturated NaCl aqueous solution (40 mL×3), respectively. The organic phase was dried with anhydrous Na₂SO₄, and filtrated. Concentrated under vacuum to get compound 4 used directly in the next step (4.9 g, yellow oil).

Step 4: Compound 4 (4.9 g, 6.43 mmol), Boc-Val (2.1 g, 9.64 mmol), DCC (1.59 g, 7.71 mmol), catalytic amount of DMAP were dissolved in CH₂Cl₂ (25 mL) and stirred overnight at room temperature. The mixture was then filtrated and the filtrate was evaporated to give a crude product which was purified by column chromatography (PE/EtOAc=10:1-5:1) to compound 5 as white solid 2.8 g, (54.5%).

Step 5: To a solution of compound 5 (2.8 g, 3.5 mmol) in methanol (25 mL) was added catalytic amount of p-toluenesulfonic acid and then the solution was stirred for overnight at room temperature. The mixture was evaporated to remove the solvent, and the residue was dissolved in EtOAc (35 mL). The solution was washed with saturated NaHCO3 aqueous solution (40 mL×3) and saturated NaCl aqueous solution (40 mL×3), respectively. The organic phase was dried with anhydrous Na2SO4, and filtrated, concentrated under vacuum to get compound 6 used directly in next step (2.57 g, white solid).

Step 6: Dry hydrogen chloride gas was bubbled into a solution of compound 6 (2 g, 2.8 mmol) in dry ether for overnight. The precipitate was collected by filtration and washed with cold dry ether to give compound 7 (1.87 g, light yellow solid). ESI-MS m/z 615.1 [M+H], ¹HNMR (CDCl3): δ 8.91 (s, 3H), 5.23 (s, 1H), 5.17˜5.19 (m, 1H), 4.08 (s, 1H), 3.88-3.90 (m,1H), 3.19˜3.24 (m, 1H), 2.46˜2.49 (m, 1H), 2.18˜2.22 (d, 1H), 1.33˜2.02 (m, 28H), 1.18 (s, 6H), 1.08 (s, 3H), 0.99 (s, 3H), 0.95 (s, 3H), 0.92 (s, 3H), 0.84˜0.87 (d, 3H, J=8.4 Hz), 0.78 (s, 3H), 0.73 (s, 3H).

Step 7: Compound 7 (500 mg, 0.77 mmol), Boc-Val (200 mg, 0.92 mmol), DCC (190 mg, 0.92 mmol), catalytic amount of DMAP were dissolved in CH₂Cl₂ (25 mL) and stirred overnight at room temperature. The mixture was then filtrated and the filtrate was evaporated to give a crude product which was purified by column chromatography (PE/EtOAc=8:1) to get compound 8 (white solid, 190 mg. 29.0%).

Step 8: Dry hydrogen chloride gas was bubbled into a solution of compound 8 (190 mg, 0.22 mmol) in dry ether for overnight. The precipitate was collected by filtration and washed with cold dry ether to give compound 9 (white solid, 160 mg, 92.4%). ESI-MS m/z 714.1 [M+H], ¹HNMR (CDCl3): δ 8.37 (s, 3H), 5.24 (s, 1H), 5.17 (m, 1H), 4.37˜4.40 (m, 1H), 3.97-4.08 (m, 2H), 3.20˜3.24 (m, 1H), 1.26˜2.44 (m, 39H), 1.09 (s, 3H), 1.00 (s, 3H), 0.96 (s, 3H), 0.92 (s, 3H), 0.86 (s, 3H), 0.79 (s, 3H), 0.74 (s, 3H).

The following compounds were prepared using a procedure similar to that described above.

| No. | Structure | NMR/MS |
|---|---|---|
| 1 | 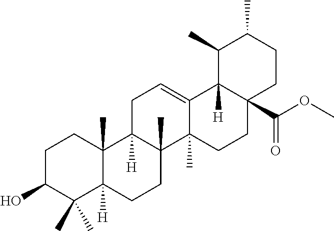 | ¹H NMR (300 MHz, CDCl₃) δ 5.15~5.18 (t, 1H, J = 4.8Hz), 3.53 (s, 3H), 3.11-3.16 (m, 1H), 2.13~2.17 (m, 1H), 1.18~1.98 (m, 23H), 1.01 (s, 3H), 0.92 (s, 3H), 0.86~0.88 (d, 3H), 0.85 (s, 3H), 0.77~0.79 (d, 3H, J = 8.4Hz), 0.71 (s, 3H), 0.67 (s, 3H). ESI-MS m/z 47.12 [M + H]. |
| 2 | 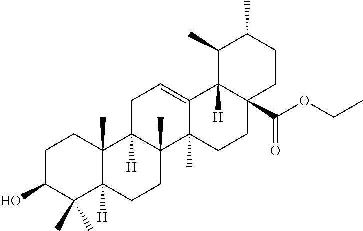 | ¹H NMR (300 MHz, CDCl₃) δ 5.15~5.17 (t, 1H, J = 4.8Hz), 3.94~4.01 (q, 2H, J = 9.4Hz), 3.12-3.16 (m, 1H), 2.13~2.17 (m, 1H), 1.18~1.98 (m, 23H), 1.12~1.17 (t, 3H, J = 9.4Hz), 1.00 (s, 3H), 0.92 (s, 3H), 0.86~0.88 (d, 3H), 0.85 (s, 3H), 0.77~0.79 (d, 3H, J = 8.4Hz), 0.71 (s, 3H), 0.69 (s, 3H). ESI-MS m/z 485.2 [M + H]. |
| 3 | 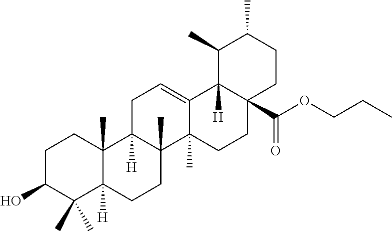 | ESI-MS m/z 499.2 [M + H]. |
| 4 | 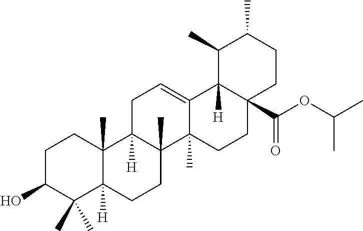 | ¹H NMR (300 MHz, CDCl₃) δ 5.16~5.18 (t, 1H, J = 4.8Hz), 4.80~4.88 (m, 1H), 3.11-3.17 (m, 1H), 2.13~2.17 (m, 1H), 1.18~1.98 (m, 23H), 1.09~1.14 (q, 6H), 1.01 (s, 3H), 0.92 (s, 3H), 0.86~0.88 (d, 3H), 0.85 (s, 3H), 0.77~0.79 (d, 3H, J = 8.4Hz), 0.71 (s, 3H), 0.71 (s, 3H). ESI-MS m/z 499.2 [M + H]. |
| 5 | 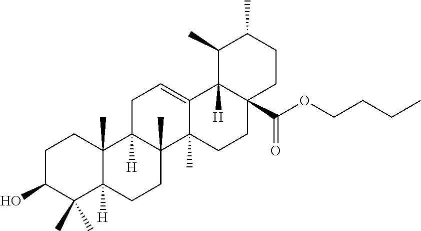 | ESI-MS m/z 513.3 [M + H]. |
| 6 | 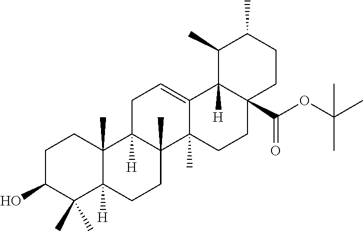 | ESI-MS m/z 513.3 [M + H]. |
| 7 | 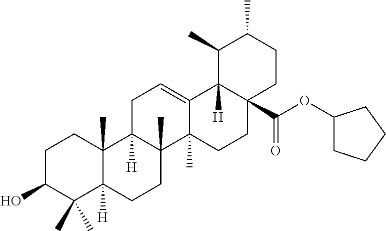 | ESI-MS m/z 525.3 [M + H]. |
| 8 | 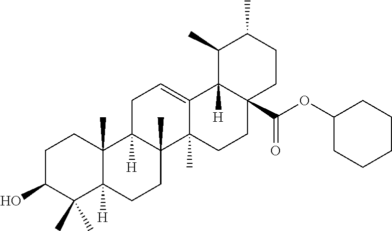 | ESI-MS m/z 539.3 [M + H]. |
| 9 | 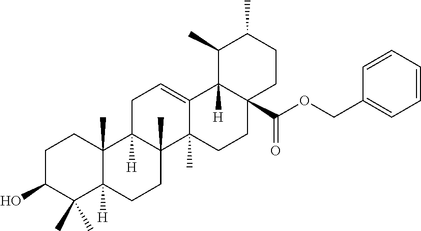 | ¹H NMR (300 MHz, CDCl₃) δ 7.30 (m, 5H), 5.19~5.21 (t, 1H, J = 4.8Hz), 4.92~5.10 (q, 2H), 3.11-3.18 (m, 1H), 2.13~2.17 (m, 1H), 1.18~1.98 (m, 23H), 1.01 (s, 3H), 0.92 (s, 3H), 0.86~0.88 (d, 3H), 0.85 (s, 3H), 0.77~0.79 (d, 3H, J = 8.4Hz), 0.71 (s, 3H), 0.67 (s, 3H). ESI-MS m/z 547.3 [M + H]. |
| 10 | 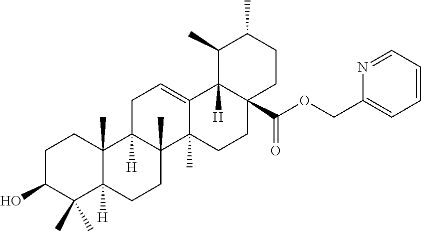 | ESI-MS m/z 548.3 [M + H]. |
| 11 | 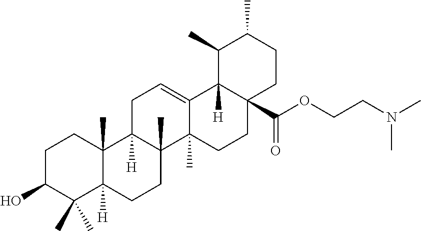 | ¹H NMR (300 MHz, CDCl₃) δ 5.22~5.24 (t, 1H, J = 4.8Hz), 4.12~4.16 (t, 2H, J = 8.0Hz), 3.19-3.24 (m, 1H), 2.58~2.61 (t, 2H, J = 8Hz), 2.32 (s, 6H), 2.20~2.24 (m, 1H), 1.25~2.04 (m, 23H), 1.08 (s, 3H), 0.99 (s, 3H), 0.93~0.95 (d, 3H), 0.92 (s, 3H), 0.85~0.87 (d, 3H, J = 8.4Hz), 0.79 (s, 3H), 0.76 (s, 3H). ESI-MS m/z 528.3 [M + H]. |
| 12 | 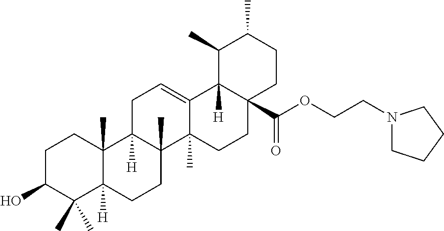 | ESI-MS m/z 554.3 [M + H]. |
| 13 | 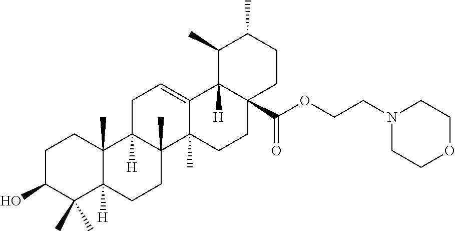 | ¹H NMR (300 MHz, CDCl₃) δ 5.15~5.17 (t, 1H, J = 4.8Hz), 4.04~4.08 (t, 2H, J = 8.0Hz), 3.62~3.65 (t, 2H, J = 6.0Hz), 3.12-3.17 (m, 1H), 2.52~2.56 (t, 2H, J = 8.0Hz), 2.42~2.45 (t, 2H, J = 6.0Hz), 2.12~2.16 (m, 1H), 1.19~1.97 (m, 23H), 1.00 (s, 3H), 0.93 (s, 3H), 0.86~0.88 (d, 3H), 0.85 (s, 3H), 0.78~0.80 (d, 3H, J = 8.4Hz), 0.72 (s, 3H), 0.69 (s, 3H). ESI-MS m/z 570.3 [M + H]. |
| 14 | 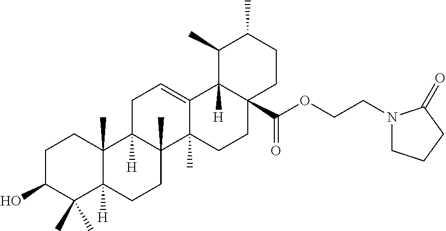 | ESI-MS m/z 568.3 [M + H]. |
| 15 | 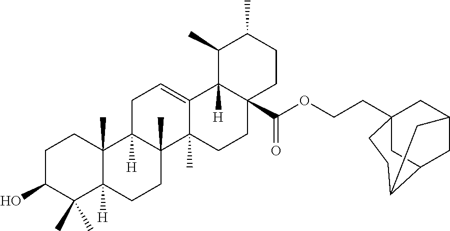 | ESI-MS m/z 619.4 [M + H]. |
| 16 | 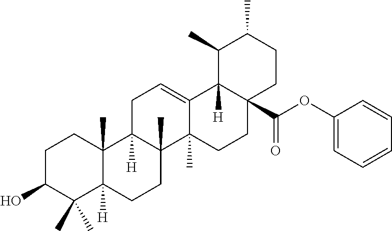 | ESI-MS m/z 533.3 [M + H]. |
| 17 | 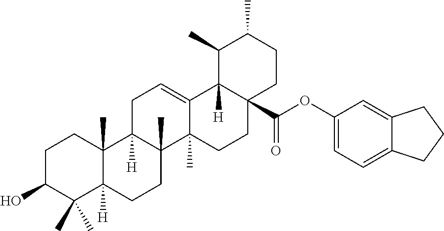 | ¹H NMR (300 MHz, CDCl₃) δ 7.14~7.16 (d, 1H, J = 10.4Hz), 6.84 (s, 1H), 6.71~6.74 (dd, 1H, J = 10.4Hz), 5.30~5.31 (t, 1H, J = 4.8Hz), 3.20-3.25 (m, 1H), 2.84~2.91 (q, 4H, J = 9.6Hz), 2.33~2.37 (m, 1H), 1.20~2.15 (m, 25H), 1.13 (s, 3H), 1.00 (s, 3H), 0.97~0.99 (d, 3H), 0.94 (s, 3H), 0.88~0.90 (d, 3H, J = 8.4Hz), 0.90 (s, 3H), 0.79 (s, 3H). ESI-MS m/z 573.3 [M + H]. |
| 18 | 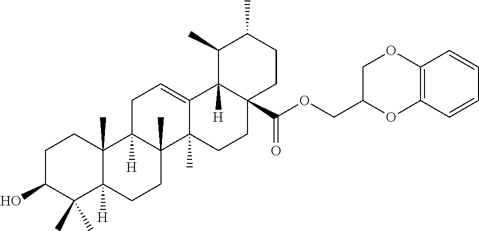 | ESI-MS m/z 605.4 [M + H]. |
| 19 | 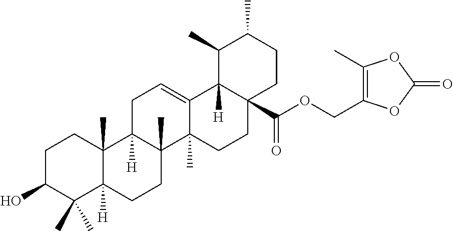 | ¹H NMR (300 MHz, CDCl₃) δ 5.25~5.26 (t, 1H, J = 4.8Hz), 4.67~4.87 (q, 2H), 3.18-3.24 (m, 1H), 2.19~2.24 (m, 1H), 2.15~2.16 (d, 3H), 1.18~1.98 (m, 23H), 1.08 (s, 3H), 0.99 (s, 3H), 0.86~0.88 (d, 3H), 0.85 (s, 3H), 0.77~0.79 (d, 3H, J = 8.4Hz), 0.79 (s, 3H), 0.65 (s, 3H). ESI-MS m/z 569.4 [M + H]. |
| 20 | 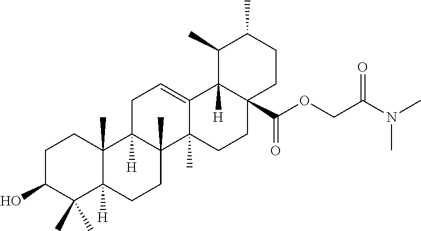 | ¹H NMR (300 MHz, CDCl₃) δ 5.16~5.18 (t, 1H, J = 4.8Hz), 4.51~4.62 (q, 2H), 3.11-3.18 (m, 1H), 2.90 (s, 3H), 2.88 (s, 3H), 2.13~2.17 (m, 1H), 1.18~1.98 (m, 23H), 1.01 (s, 3H), 0.92 (s, 3H), 0.86~0.88 (d, 3H), 0.85 (s, 3H), 0.77~0.79 (d, 3H, J = 8.4Hz), 0.71 (s, 3H), 0.67 (s, 3H). ESI-MS m/z 542.3 [M + H]. |
| 21 | 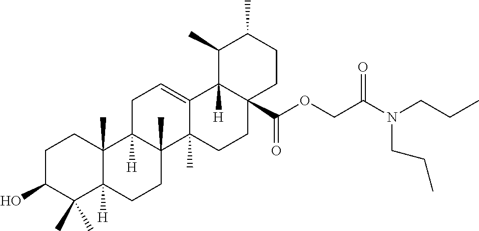 | ESI-MS m/z 598.4 [M + H]. |
| 22 | 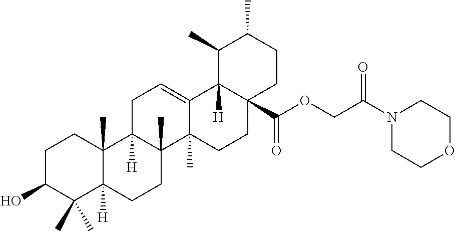 | ¹H NMR (300 MHz, CDCl₃) δ .16~5.18 (t, 1H, J = 4.8Hz), 4.51~4.62 (q, 2H), 3.5-3.6 (m, 8H), 3.11-3.18 (m, 1H) 2.90 (s, 3H), 2.88 (s, 3H), 2.13~2.17 (m, 1H), 1.18~1.98 (m, 23H), 1.01 (s, 3H), 0.92 (s, 3H), 0.86~0.88 (d, 3H), 0.85 (s, 3H), 0.7~0.79 (d, 3H, J = 8.4Hz). ESI-MS m/z 584.4 [M + H]. |
| 23 | 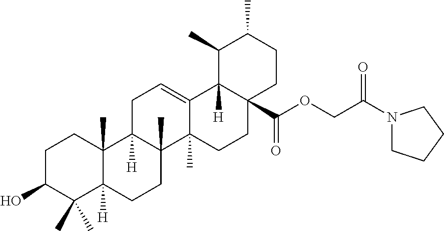 | ESI-MS m/z 568.4 [M + H]. |
| 24 | 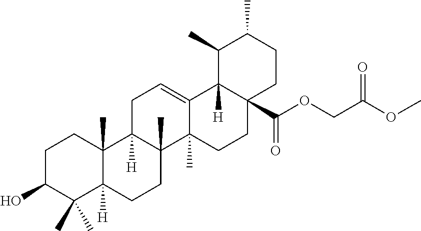 | ESI-MS m/z 529.3 [M + H]. |
| 25 | 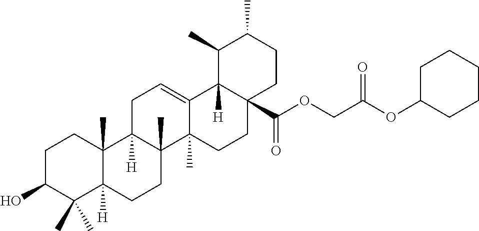 | ESI-MS m/z 597.4 [M + H]. |
| 26 | 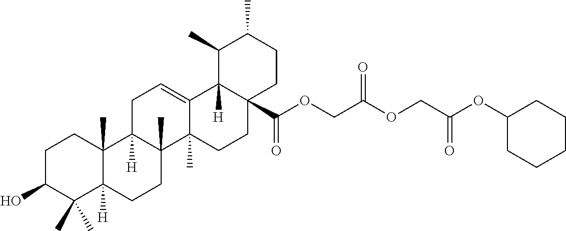 | ESI-MS m/z 655.4 [M + H]. |
| 27 | 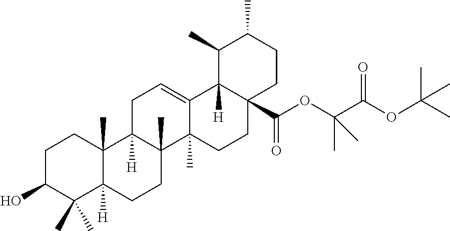 | ESI-MS m/z 599.4 [M + H]. |
| 28 | 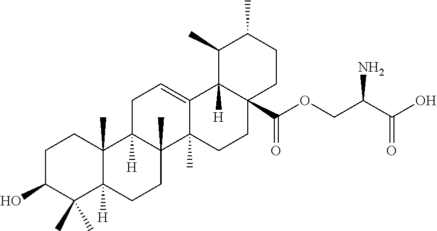 | ESI-MS m/z 544.3 [M + H]. |
| 29 | 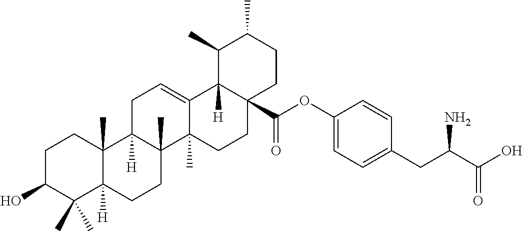 | ESI-MS m/z 620.4 [M + H]. |
| 30 | 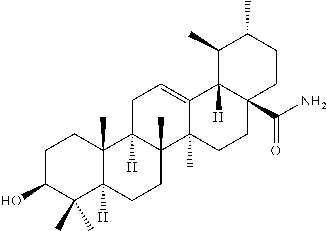 | ESI-MS m/z 456.2 [M + H]. |
| 31 | 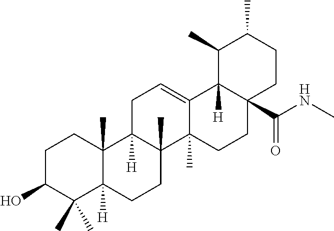 | ESI-MS m/z 470.3 [M + H]. |
| 32 | 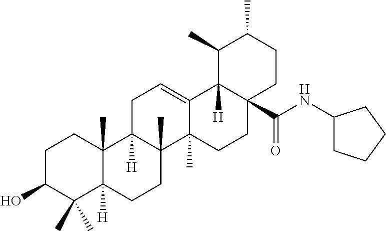 | ESI-MS m/z 524.3 [M + H]. |
| 33 | 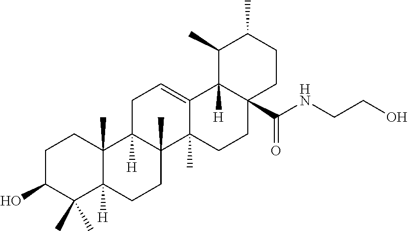 | ESI-MS m/z 500.3 [M + H]. |
| 34 | 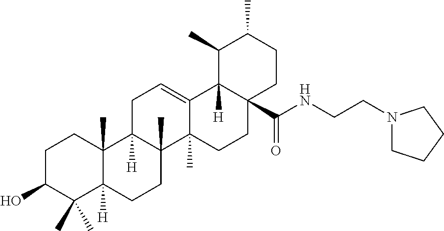 | ESI-MS m/z 553.3 [M + H]. |
| 35 | 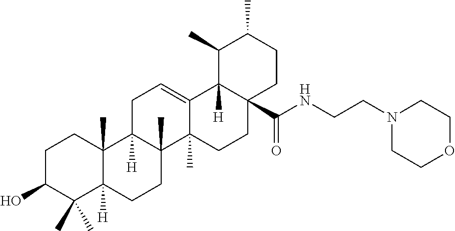 | ESI-MS m/z 570.3 [M + H]. |
| 36 | 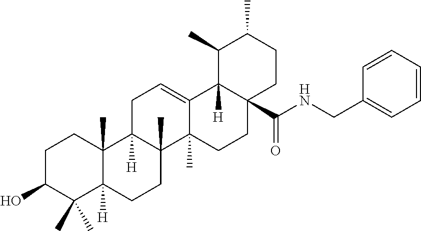 | ¹H NMR (300 MHz, CDCl₃) δ 7.26~7.33 (m 5H), 6.14~6.18 (m, 1H), 5.21~5.23 (m, 1H), 4.12~4.61 (m, 2H), 3.20-3.24 (m, 1H), 1.28~2.06 (m, 24H), 1.10 (s, 3H), 1.00 (s, 3H), 0.96 (s, 3H), 0.90 (s, 3H), 0.85~0.87 (d, 3H, J = 8.8Hz), 0.80 (s, 3H), 0.72 (s, 3H). ESI-MS m/z 546.3 [M + H]. |
| 37 | 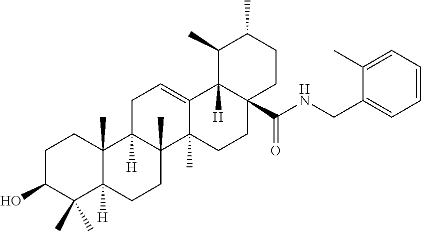 | ESI-MS m/z 560.3 [M + H]. |
| 38 | 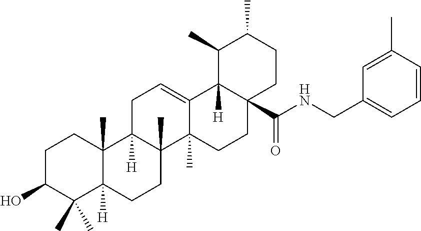 | ESI-MS m/z 560.3 [M + H]. |
| 39 | 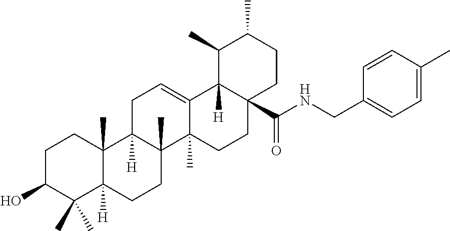 | ESI-MS m/z 560.3 [M + H]. |
| 40 | 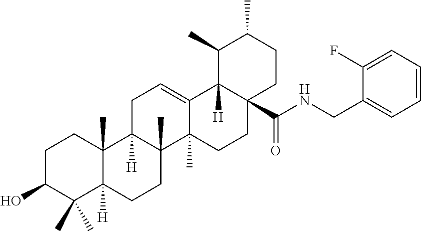 | ESI-MS m/z 564.3 [M + H]. |
| 41 | 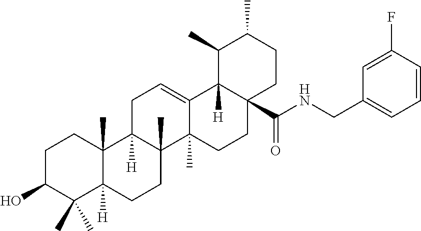 | ESI-MS m/z 564.3 [M + H]. |
| 42 | 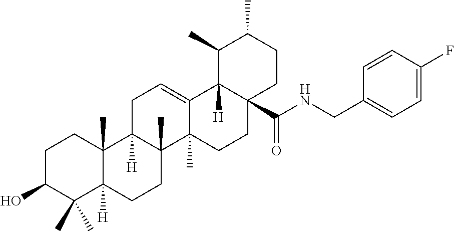 | ESI-MS m/z 564.3 [M + H]. |
| 43 | 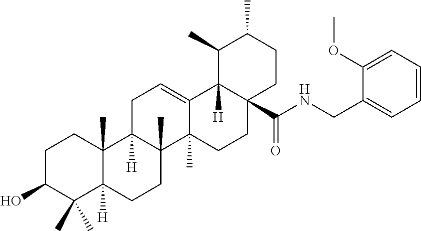 | ESI-MS m/z 576.3 [M + H]. |
| 44 | 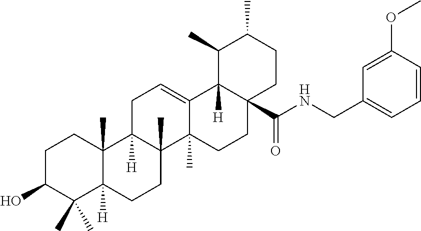 | ESI-MS m/z 576.3 [M + H]. |
| 45 | 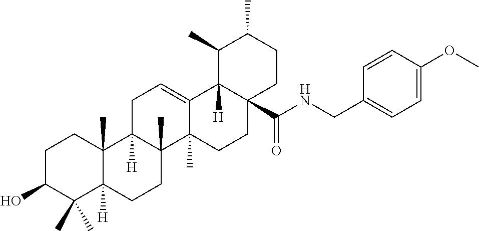 | ESI-MS m/z 576.3 [M + H]. |
| 46 | 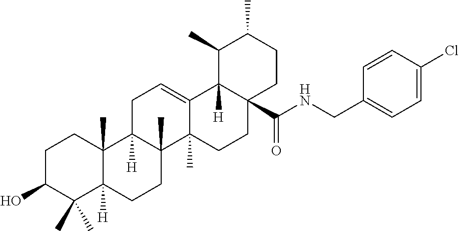 | ESI-MS m/z 580.3 [M + H]. |
| 47 | 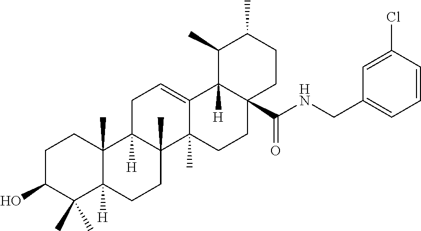 | ESI-MS m/z 580.3 [M + H]. |
| 48 | 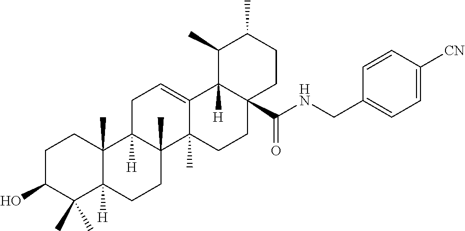 | ESI-MS m/z 571.3 [M + H]. |
| 49 | 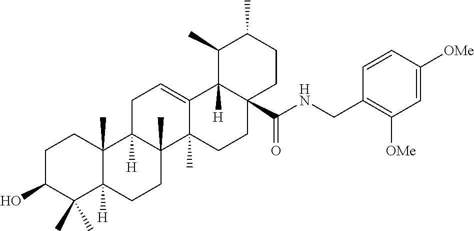 | ESI-MS m/z 606.4 [M + H]. |
| 50 | 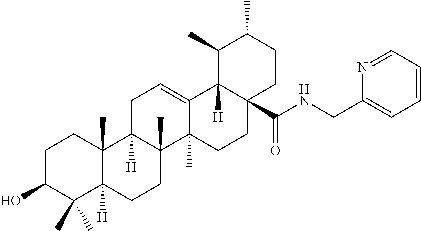 | ESI-MS m/z 547.3 [M + H]. |
| 51 | 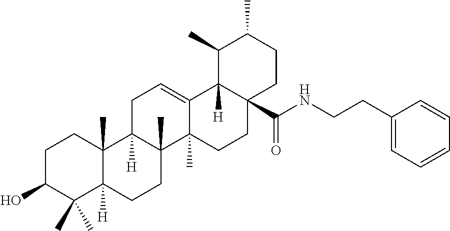 | ¹H NMR (300 MHz, CDCl₃) δ 7.18~7.33 (m, 5H), 5.83~5.88 (m, 1H), 4.89 (s, 1H), 3.72-3.81 (m, 1H), 3.14~3.23 (m, 2H), 2.69~2.89 (m, 2H), 1.20~2.01 (m, 24H), 0.96 (s, 3H), 0.90 (s, 3H), 0.86 (s, 3H), 0.80 (s, 3H), 0.72~0.74 (d, 3H, J = 8.8Hz), 0.71 (s, 3H), 0.54 (s, 3H). ESI-MS m/z 560.3 [M + H]. |
| 52 | 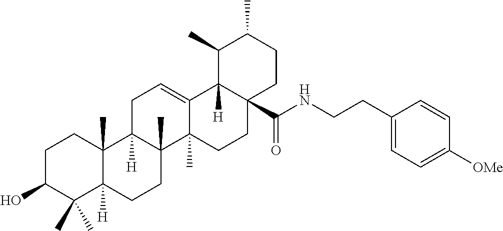 | ¹H NMR (300 MHz, CDCl₃) δ 7.01~7.04 (d, 2H, J = 11.6Hz), 6.77~6.80 (d, 2H, J = 11.6Hz), 5.78~5.80 (m, 1H), 4.81~4.83 (m, 1H), 3.73 (s, 1H), 3.60~3.71 (m, 1H), 3.10-3.15 (m, 1H), 3.02-3.06 (m, 1H), 2.54-2.75 (m, 2H), 1.15~1.90 (m, 24H), 0.96 (s, 3H), 0.90 (s, 3H), 0.86 (s, 3H), 0.80 (s, 3H), 0.73~0.75 (d, 3H, J = 8.8Hz), 0.70 (s, 3H), 0.53 (s, 3H). ESI- MS m/z 590.4 [M + H]. |
| 53 | 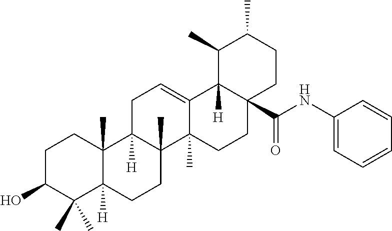 | ESI-MS m/z 532.3 [M + H]. |
| 54 | 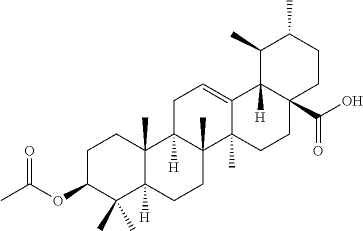 | ESI-MS m/z 499.3 [M + H]. |
| 55 | 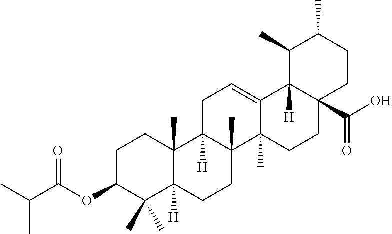 | ESI-MS m/z 527.3 [M + H]. |
| 56 | 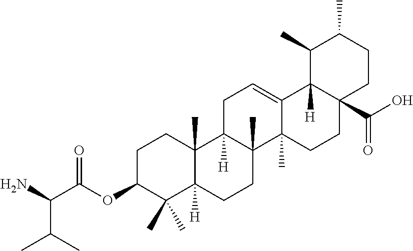 | ESI-MS m/z 556.3 [M + H]. |
| 57 | 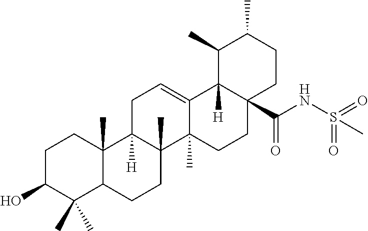 | |
| 58 | 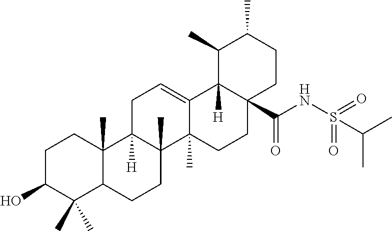 | |
| 59 | 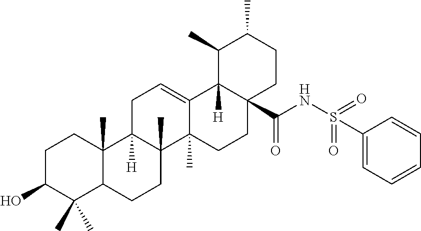 | |
| 60 | 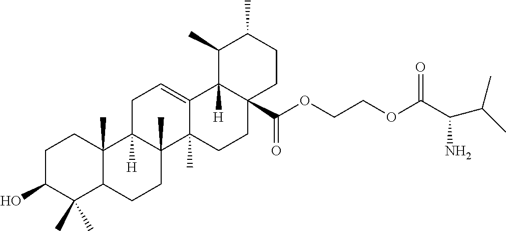 | |
| 61 | 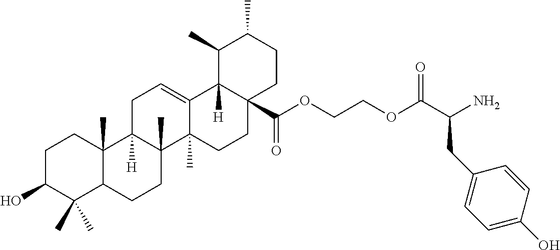 | |
| 62 | 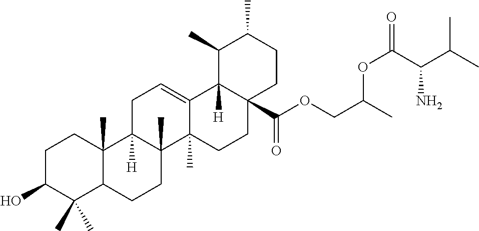 | |
| 63 | 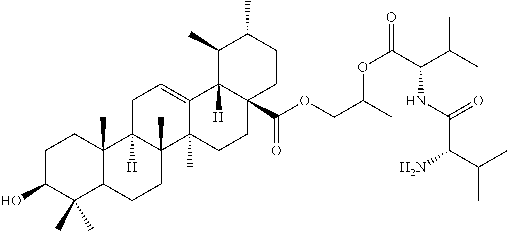 | |

### Biological Activity Assays

T-cell Isolation and Differentiation—Mouse T cell differentiation was performed and analyzed by intracellular staining as described. Xu, T., et al. Ursolic acid suppresses interleukin-17 (IL-17) production by selectively antagonizing the function of RORgamma t protein. The Journal of biological chemistry 286: 22707-22710 (2011); Wang, X., et al. Transcription of Il17 and Il17f is controlled by conserved noncoding sequence 2. Immunity 36: 23-31 (2012). Briefly, mouse naïve CD4⁺ T cells (CD4⁺CD25⁻CD62L^{hi}-CD44^{lo}) were isolated from spleens and lymph nodes by FACS sorting. Th17 differentiation was performed with 15 ng ml⁻ IL-6 and 2.5 ng ml⁻ TGF-β in the presence of 1 μg ml⁻ anti-CD3 (plate bound) and 1 μg ml⁻ anti-CD28 (plate bound) for 4 days. When indicated, different concentrations of UA, KL001, other UA analogs or small molecules (dissolved in DMSO) were added at the beginning of Th17 differentiation. The mature Th17 cells were restimulated with 50 ng ml⁻ PMA (phorbol 12-myristate 13-acetate) and 500 ng ml⁻ inomycin in the presence of BD GolgiStop™ for 6 hrs, stained intracellularly for IL-17 and IL-17F, and then analyzed by flow cytometry.

MOG-induced Experimental Autoimmune Encephalomyelitis (EAE)—EAE induction were performed by immunizing mice twice with MOG35-55 peptide (amino acids 35-55; MEVGWYRSPFSROVHLYRNGK) emulsified in CFA followed by pertussis toxin injection with slight modifications, and analyzed as previously described. Xu, T., et al. Ursolic acid suppresses interleukin-17 (IL-17) production by selectively antagonizing the function of RORgamma t protein. The Journal of biological chemistry 286: 22707-22710 (2011); Wang, X., et al. Transcription of Il17 and Il17f is controlled by conserved noncoding sequence 2. Immunity 36: 23-31 (2012). Briefly, the disease scores were assigned on a scale of 0-5 as follows: 0, none; 1, limp tail or waddling gait with tail tonicity; 2, wobbly gait; 3, hindlimb paralysis; 4, hindlimb and forelimb paralysis; 5, death. In the preventative EAE model, UA and KL001 were dissolved in DMSO, and given to mice at a dose of ˜100 mg/Kg body weight by intraperitoneal injection (i.p.) every other day after first MOG immunization; In the therapeutic EAE model, UA and KL001 were dissolved in 0.5% MC (methyl cellulose), and given to mice at a dose of ˜250 mg/Kg body weight by gavage twice daily after the second MOG immunization.

IMQ-induced psoriasis model—The mouse psoriasis model was induced according to previous described with minor modifications. van der Fits L, et al. (2009) Imiquimod-induced psoriasis-like skin inflammation in mice is mediated via the IL-23/IL-17 axis. J Immunol 182(9):5836-5845. Briefly, 7-9 weeks old female C57BL/6 mice were used to induce psoriasis. On day 0, the hair was removed from the dorsal skin of the mice after anaesthetizing with pentobarbital (˜0.2 ml/20 g body weight by i.p.). On day 1, psoriasis was induced at day 3 by applying 62.5 mg 5% imiquimod cream to the dorsal skin after anaesthesia on a daily base. In the preventative disease model, KL001 (dissolved in 0.5% methylcellulose (MC)) or 0.5% MC (control group) was orally administrated into C57BL/6 mice by gavage at a dosage of ˜250 mg/kg body weight twice daily starting 2 days before induction of psoriasis (Day −2). The mice were then sacrificed on Day 5, and the skin tissues were then removed for H&E staining and mRNA expression analysis. For mRNA expression analysis, total RNA was prepared from the skin samples with TriZol reagent (Invitrogen) after homogenization, and cDNA was synthesized with reverse transcriptase and oligo(dT) primers. The expression of Il17, Il17f and Rorc (encoding RORγt) were then quantified by realtime PCR and normalized to Actb gene by using the following primers: Actb, F: TGGAATCCTGTGGCATCCATGAAAC/R (SEQ ID NO.1): TAAAACGCAGCTCAGTAACAGTCCG (SEQ ID NO.2); Il17,F: CTCCAGAAGGCCCTCAGACTAC/R (SEQ ID NO.3): GGGTCTTCATTGCGGTGG (SEQ ID NO.4); Il17f, F: CCCATGGGATTACAACATCACTC/R (SEQ ID NO.5): CACTGGGCCTCAGCGATC (SEQ ID NO.6); Rorc, F: CCGCTGAGAGGGCTTCAC/R (SEQ ID NO.7): TGCAGGAGTAGGCCACATTACA (SEQ ID NO.8).

Calculations and Statistic Analysis. Most in vitro and in vivo data were repeated for 2-3 times with consistent results. When indicated, the statistic significance was determined by student's t-test. (* represents P<0.05; ** represents P<0.03; *** represents P<0.01).

### Results

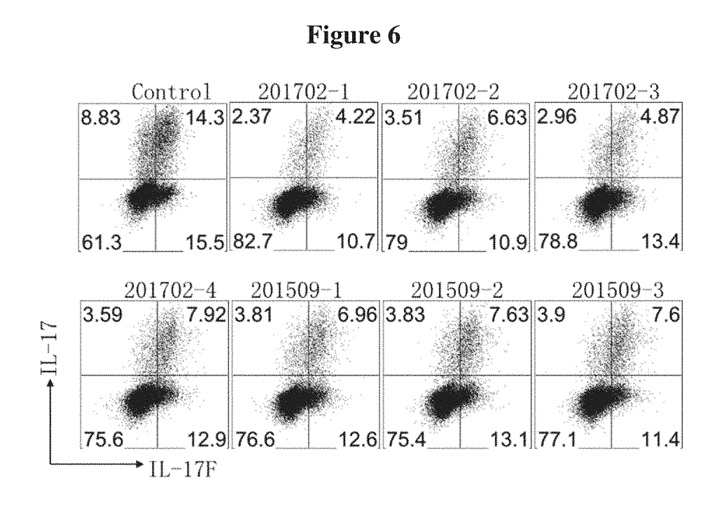

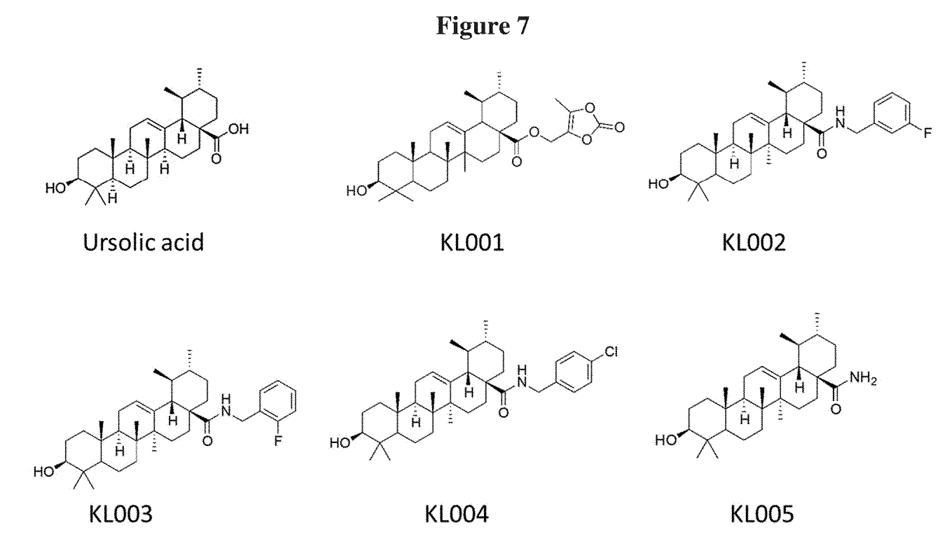

UA and its analogs KL001 to KL005, 201702-1 to 201702-4, and 201509-1 to 201509-3 inhibit TH17 differentiation. Mouse naïve CD4+ T cells were cultured under Th17 polarizing condition in the presence of above chemicals (2-10 μM) or DMSO control (Ctrl) for 4 days and then restimulated for IL-17 and IL-17F staining (FIG. 1). Consistent with previous findings, Xu, T., et al. Ursolic acid suppresses interleukin-17 (IL-17) production by selectively antagonizing the function of RORgamma t protein. The Journal of biological chemistry 286: 22707-22710 (2011), 2 μM UA inhibited ˜80% IL-17 expression and ˜50% IL-17F expression, both of which were reported to contribute TH17-mediated autoimmune diseases Yang, X. O., Chang, S. H., Park, H. et al. Regulation of inflammatory responses by IL-17F. The Journal of experimental medicine, 205: 1063-1075 (2008). Interestingly, it was found that several UA derived analogs, including KL001, KL002 and KL005 inhibited TH17 differentiation at comparable level to UA. (FIG. 1), whereas other UA analogs inhibited TH17 differentiation at a less degree (FIG. 1 and FIG. 6). Moreover, a titration of inhibitory assay showed the IC50 value of KL001 is ˜0.49 μM in inhibiting Th17 differentiation, as determined by the readout of IL-17 staining data (FIG. 2), and this is comparable to that of UA (IC50=0.68 μM) as examined in our previous studies, Xu, T., et al. Ursolic acid suppresses interleukin-17 (IL-17) production by selectively antagonizing the function of RORgamma t protein. The Journal of biological chemistry 286: 22707-22710 (2011), further demonstrating that KL001 released UA as the major active ingredient in inhibiting Th17 differentiation.

KL001 showed better efficacy than UA in reducing EAE in a preventative disease model: Previous studies showed that UA can effectively ameliorate EAE in a preventative disease model in mice. Xu, T., et al. Ursolic acid suppresses interleukin-17 (IL-17) production by selectively antagonizing the function of RORgamma t protein. The Journal of biological chemistry 286: 22707-22710 (2011). To test the in vivo effect of UA derived analogs, EAE was induced in mice by MOG immunization and PTX injection as described. Xu, T., et al. Ursolic acid suppresses interleukin-17 (IL-17) production by selectively antagonizing the function of RORgamma t protein, The Journal of biological chemistry 286: 22707-22710 (2011); Wang, X., et al. Transcription of Il17 and Il17f is controlled by conserved noncoding sequence 2, Immunity 36: 23-31 (2012). UA (n=4), KL001 (n=3) and DMSO (n=5) were given to mice by IP injection every other day at a dosage of 100 mg/kg body weight after first MOG immunization. The results showed that KL001 inhibited EAE at greater degree than UA in this preventative EAE disease model (FIG. 3). In addition, the T cells infiltrated into the central nerve system of EAE mice showed significantly reduced IL-17 expression upon either UA or KL001 treatment (FIG. 3). These results show that KL001 is better than UA in inhibiting TH17 differentiation and TH17-induced autoimmunity.

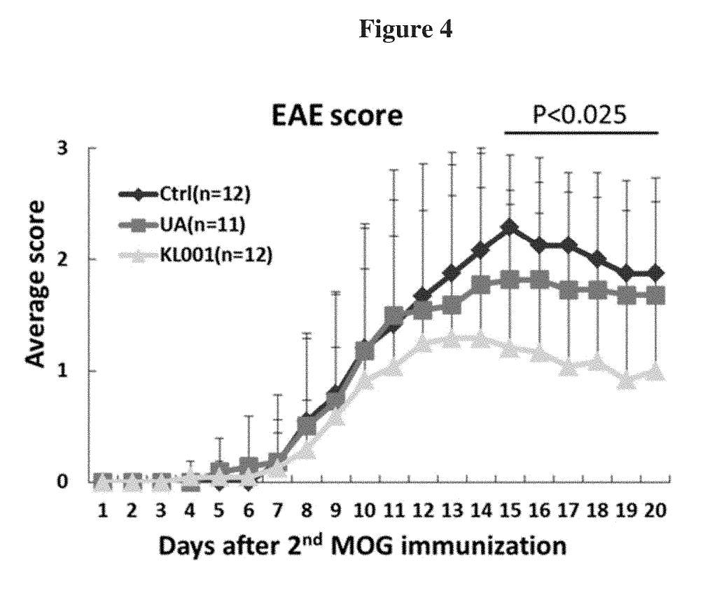

KL001 showed better efficacy in treating EAE in a therapeutic disease model: As stated above, KL001 was able to inhibit TH17 differentiation in vitro and in vivo, and KL001 showed better efficacy in inhibiting TH17-mediated autoimmunity in a preventative disease model. To test the therapeutic potential of KL001, EAE was first induced by MOG immunization and PTX injection as described. Xu, T., et al. Ursolic acid suppresses interleukin-17 (IL-17) production by selectively antagonizing the function of RORgamma t protein, The Journal of biological chemistry 286: 22707-22710 (2011); Wang, X., et al. Transcription of Il17 and Il17f is controlled by conserved noncoding sequence 2, Immunity 36: 23-31 (2012). At Day 9 after the first MOG immunization when the TH17 response was fully induced in vivo, UA (n=11), KL001 (n=12) or control vehicle (0.5% methyl cellulose) (n=12) were given to mice twice daily by gavage administration at a dosage of ˜250 mg/kg body weight. Although UA treatment did not affect the EAE disease, KL001 administration significantly reduced the disease severity of EAE, and the average disease scores were reduced by 50% when compared with the control group (FIG. 4). Taken together, these results show that the UA-derived analogs, such as KL001, were able to inhibit TH17 differentiation in vitro and in vivo, as well as to ameliorate EAE disease in mice in both preventative and therapeutic disease models. These findings demonstrate that KL001 or other UA-derived analogs can be used as potential drug candidates in the clinical application in treatment of various TH17- or IL-17-related inflammation and autoimmune diseases.

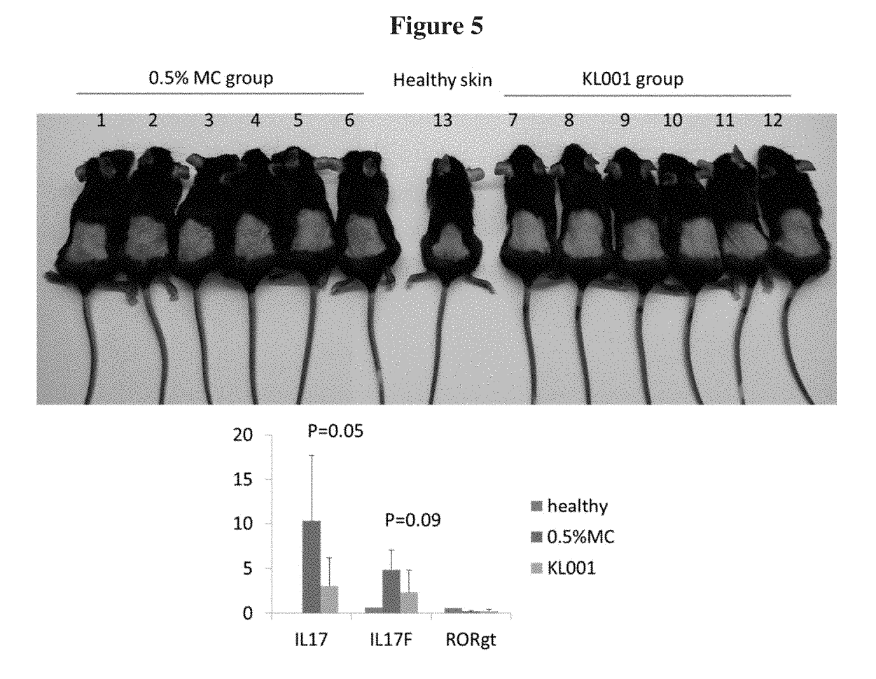

KL001 effectively ameliorated IMQ-induced psoriasis in a preventative disease model: Aberrant Th17 responses are known to cause many chronic inflammatory and autoimmune diseases, including multiple sclerosis, psoriasis, rheumatoid arthritis, inflammatory bowel diseases, periodontal diseases and asthma airway inflammatory diseases. Korn, T., et al., IL-17 and Th17 Cells, Annu Rev Immunol. 27:485-517 (2009); Tesmer, L. A., et al., Th17 Cells in Human Disease, Immunol Rev. 223:87-113 (2008). In the above studies, it was showed that KL001, a small molecule derived from ursolic acid, can inhibit Th17-mediated EAE disease, a mouse model of human multiple sclerosis, in both preventative and therapeutic experimental settings. To determine whether KL001 has therapeutic potential in additional Th17-related diseases, the role of KL001 in imiquimod (IMQ)-induced mouse disease model of psoriasis was then examined. Briefly, KL001 was dissolved in 0.5% methylcellulose (MC), and then orally administrated into C57BL/C mice by gavage at a dosage of ˜250 mg/kg body weight twice daily during the process of experiment, with equal volume of 0.5% MC treatment group as the control. Psoriasis was induced at day 3 by applying 62.5 mg 5% imiquimod cream daily to the dorsal skin after anaesthetizing with pentobarbital (˜0.2 ml/20 g body weight by i.p.) and hair removal, as described previously. van der Fits L, et al. (2009) Imiquimod-induced psoriasis-like skin inflammation in mice is mediated via the IL-23/IL-17 axis. J Immunol 182(9):5836-5845. The mice were then sacrificed 3.5 days after induction of psoriasis, and the skin tissues were then removed for histology and mRNA analysis. Compared with control group, treatment of KL001 visually and greatly reduced the redness and roughness of the skin in psoriasis model (FIG. 5). In addition, the expression of IL-17, but not RORγt, was also notably reduced in the skin samples of KL001 treated mice. The success of KL001 in alleviating Th17-mediated inflammation in distinct mouse disease models such as EAE and psoriasis via targeting RORγt indicates potential applications of this and other UA analogs in treatment of various Th17- or RORγt-dependent human autoimmune diseases.

It was noted that KL001 releases UA as the major active ingredient, which in turn then targets RORγt to inhibit RORγt-directed TH17 program and TH17-related diseases. It is known that UA contains many pharmacological activities, including strong hepatoprotective, antitumor and anti-inflammation effects partly through targeting NF-κB and STAT3. Liu, J. Pharmacology of Oleanolic Acid and Ursolic Acid, J Ethnopharmacol. 49:57-68 (1995); Ikeda, Y., et al., Ursolic Acid: An Anti- and Pro-Inflammatory Triterpenoid, Mol Nutr Food Res. 52:26-42(2008); Huang, H. C., et al., Ursolic Acid Inhibits IL-1β or TNF-α-Induced C6 Glioma Invasion Through Suppressing the Association ZIP/p62 with PKC-ζ and Downregulating the MMP-9 Expression, Mol Carcinog. 48:517-531 (2009); Shishodia, S., et al., Ursolic Acid Inhibits Nuclear Factor-β Activation Induced by Carcinogenic Agents through Suppression of IκBα Kinase and p65 Phosphorylation: Correlation with Down-Regulation of Cyclooxygenase 2, Matrix Metalloproteinase 9, and Cyclin D1, Cancer Res. 63:4375-4383 (2003); Pathak, A. K., et al., Ursolic Acid Inhibits STAT3 Activation Pathway Leading to Suppression of Proliferation and Chemosensitization of Human Multiple Myeloma Cells, Mol Cancer Res. 5:943-955 (2007). In previous studies, Xu, T., et al. Ursolic acid suppresses interleukin-17 (IL-17) production by selectively antagonizing the function of RORgamma t protein. The Journal of biological chemistry 286: 22707-22710 (2011), it was demonstrated that UA has a higher affinity for RORγt (˜0.7 μM) than for NF-κB and STAT3, and 2 μM of UA effectively blocked the activity of RORγt and RORγt-direct IL-17 expression, whereas the inhibitory activity of UA on NF-κB and STAT3 was only observed when used at a concentration higher than 25 μM. In addition, PK studies suggest that the UA concentration in the blood of EAE mice receiving either UA or KL001 was far below 25 μM, demonstrating that UA and KL001 inhibit EAE through targeting RORγt rather than NF-κB and STAT3.

As a natural small molecule ubiquitously present in plants and even human diets, UA is relatively non-toxic and is well tolerated orally and topically in both human and rodents. The acute toxicity (LD50) of UA in rodents was determined to be >637 mg/kg for intraperitoneal injection and 8330 mg/kg for oral administration. Lee, A. W., et al., Ursolic Acid Induces Allograft Inflammatory Factor-1 Expression via a Nitric Oxide-Related Mechanism and Increases Neovascularization, J Agric Food Chem. 58:12941-12949 (2010). In addition, UA has been identified as a major effective component in many medical herbs which have a long history in clinic practice in ancient China and Asian countries. Liu, J. Pharmacology of Oleanolic Acid and Ursolic Acid, J Ethnopharmacol. 49:57-68 (1995). Due to its important pharmacological activities, UA has been used for treatment of liver diseases and skin cancer. Liu, J. Pharmacology of Oleanolic Acid and Ursolic Acid, J Ethnopharmacol. 49:57-68 (1995). These clinical practices and its relatively low toxicity provide UA and UA-derived analogs that release UA as the major active ingredient a great advantage over other RORγt inhibitors in developing therapeutics against Th17-mediated autoimmune diseases.

Considering the broad functionality of the Th17 cell, particularly in inflammatory diseases and cancer, the therapeutic effects of KL001 and other UA related analogs as described herein via inhibition of RORγt and ultimately Th17 cell function can serve a wide variety of therapeutic applications. Since KL001 and other UA related analogs release UA as the major active ingredient, these UA-derived analogs can be potentially used to replace UA in treating UA-treatable diseases, such as liver diseases and skin cancer. Liu, J. Pharmacology of Oleanolic Acid and Ursolic Acid, J Ethnopharmacol. 49:57-68 (1995).

What is claimed is:

1. A compound for modulating retinoid-related orphan receptor gamma (ROR-gamma), said compound has the structure of: 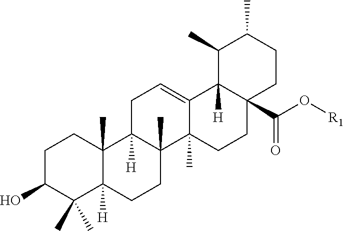
or a physiologically acceptable salt or hydrate or solvate thereof, wherein R₁ is selected from the group consisting of:

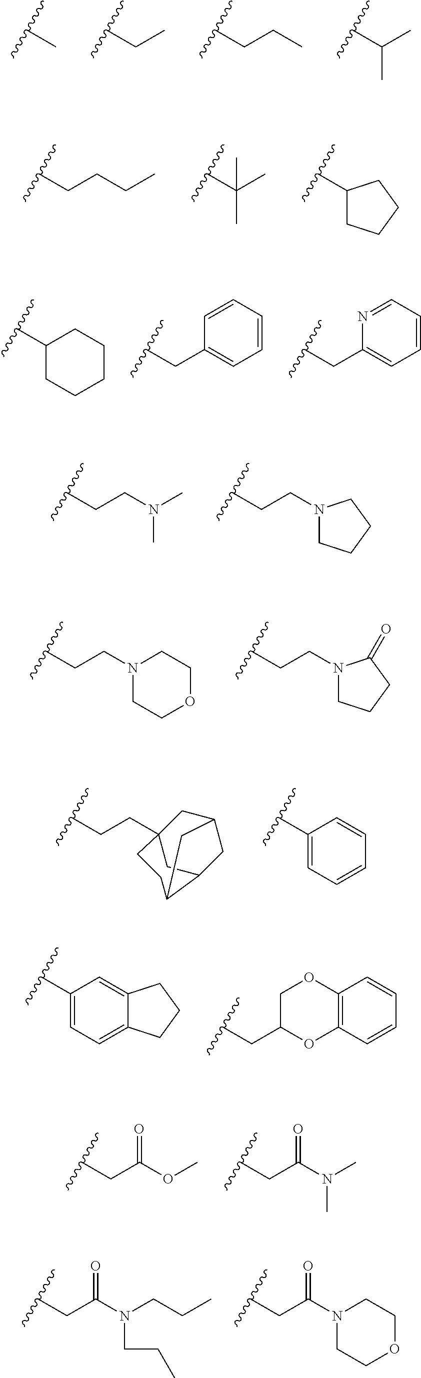

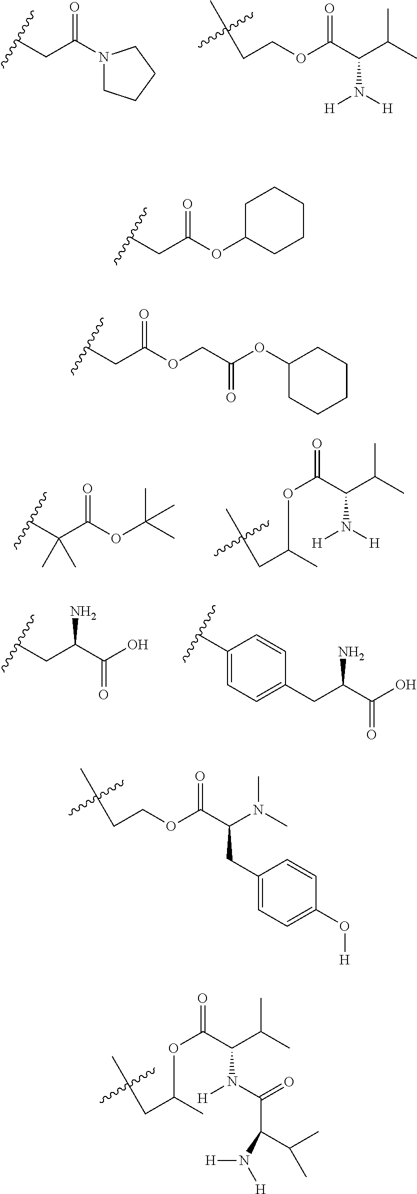

2. The compound of claim 1, wherein R₁ is selected from the group consisting of:

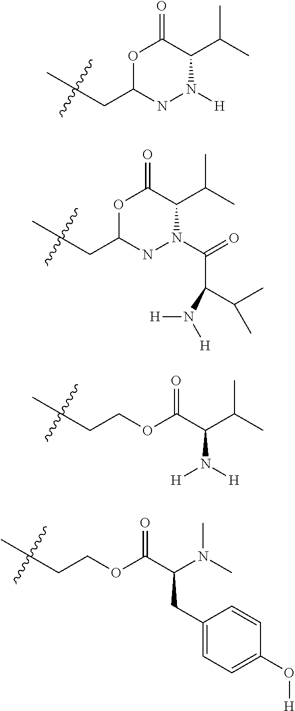

3. A composition comprising the compound of claim 1, wherein the composition comprises at least one pharmaceutically acceptable carrier or diluent.

4. The composition of claim 3, wherein the composition is in a form of a tablet, a capsule, an injectable composition, an ingestible composition, a nasal spray, an aerosol, or a suppository.

5. A compound for modulating retinoid-related orphan receptor gamma (ROR-gamma), said compound has the structure of: 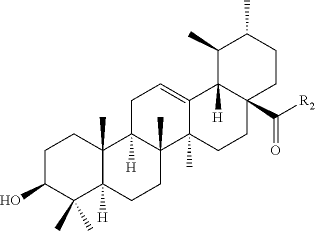
or a physiologically acceptable salt or hydrate or solvate thereof, wherein R₂ is selected from the group consisting of:

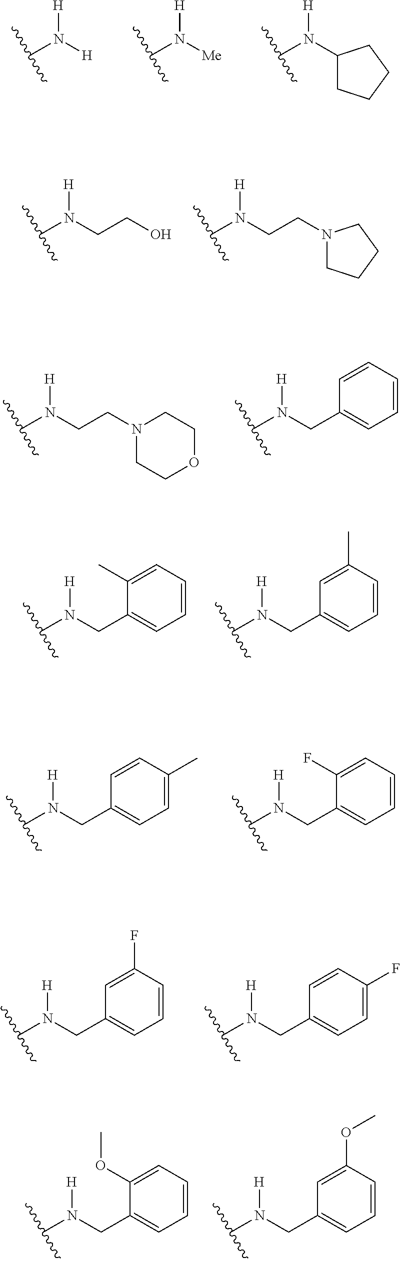

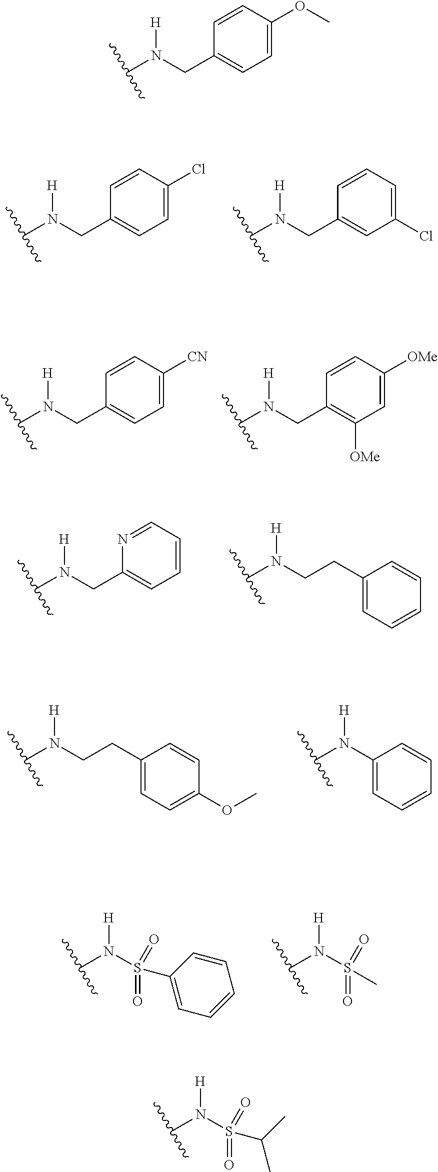

6. The compound of claim 5, wherein R₂ is selected from the group consisting of:

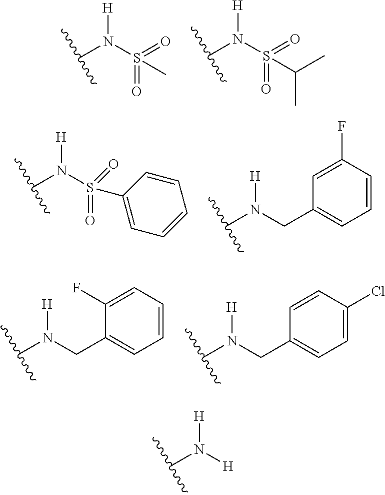

7. A composition comprising the compound of claim 5, wherein the composition comprises at least one pharmaceutically acceptable carrier or diluent.

8. The composition of claim 7, wherein the composition is in a form of a tablet, a capsule, an injectable composition, an ingestible composition, a nasal spray, an aerosol, or a suppository.

9. A compound for modulating retinoid-related orphan receptor gamma (ROR-gamma), said compound has the structure of: 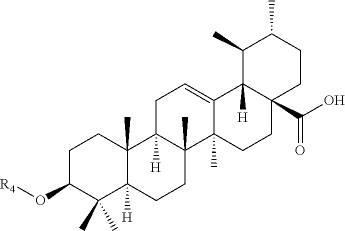
or a physiologically acceptable salt or hydrate or solvate thereof, wherein R₄ is selected from the group consisting of:

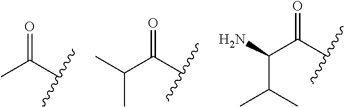

10. A composition comprising the compound of claim 9, wherein the composition comprises at least one pharmaceutically acceptable carrier or diluent.

11. A method of inhibiting differentiation of a population of T cells, comprising contacting said T cells with a composition comprising a compound having the structure of: 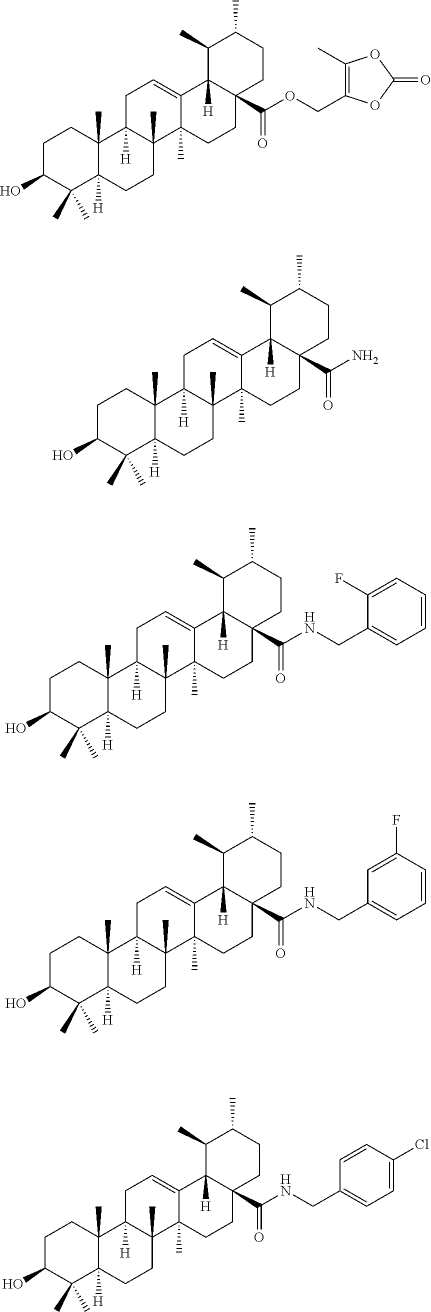 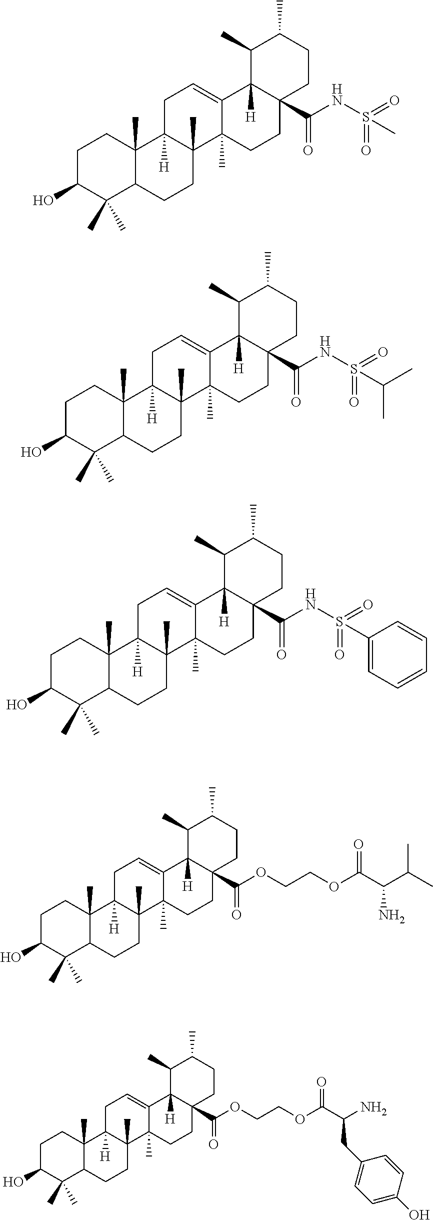 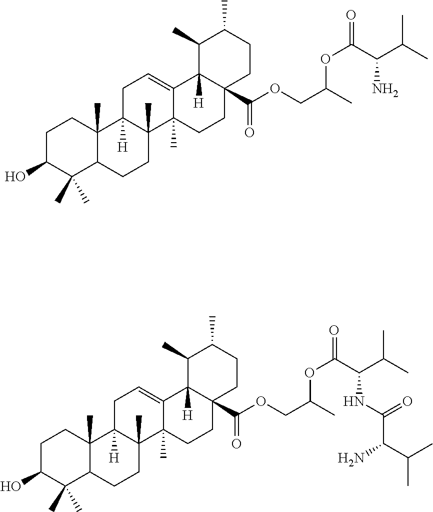
or a physiologically acceptable salt or hydrate or solvate thereof.

12. The method of claim 11, wherein said T cells are Th17 cells.

13. A method of treating a disease caused by Th17 cell activities in a subject in need of such treatment, said method comprises administering to said subject a composition comprising a therapeutically effective amount of a compound having the structure of: 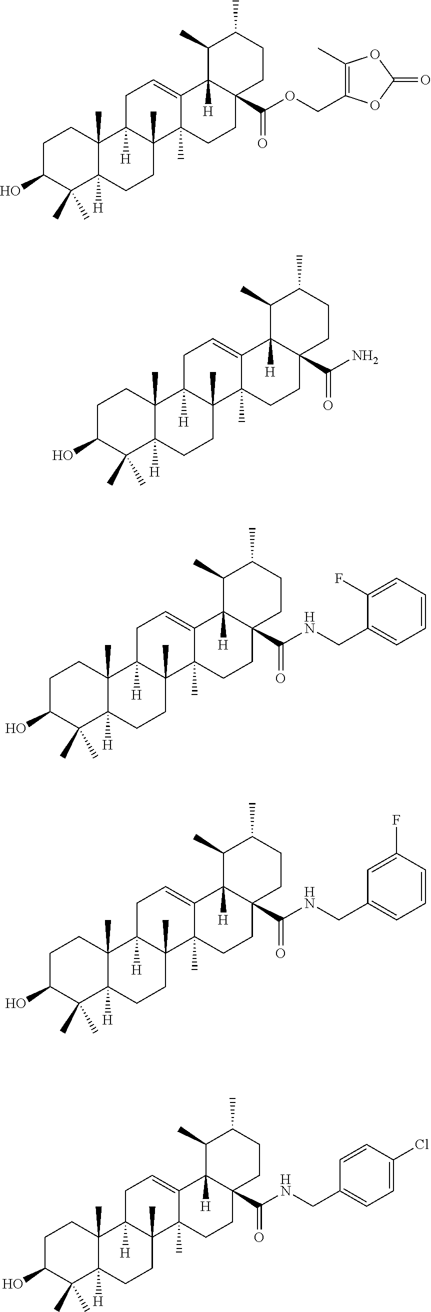 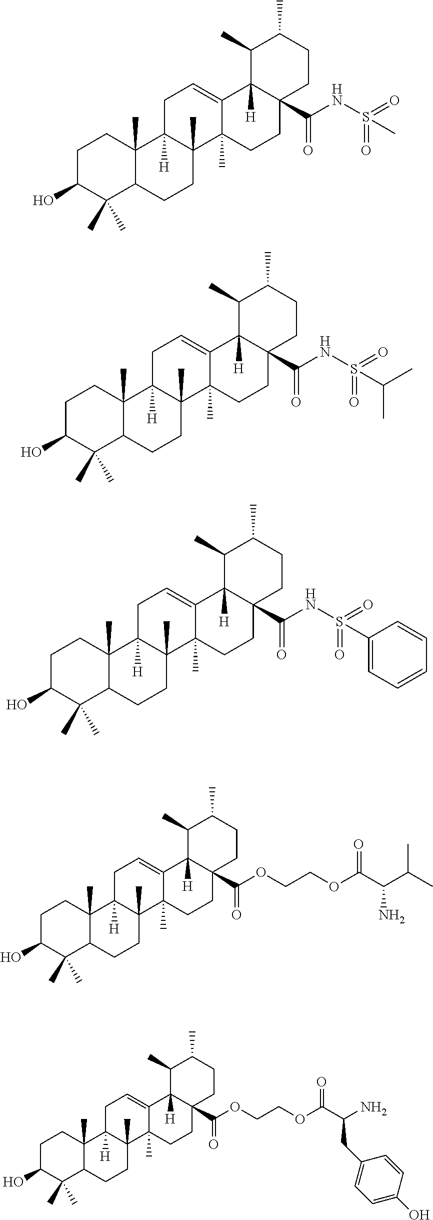 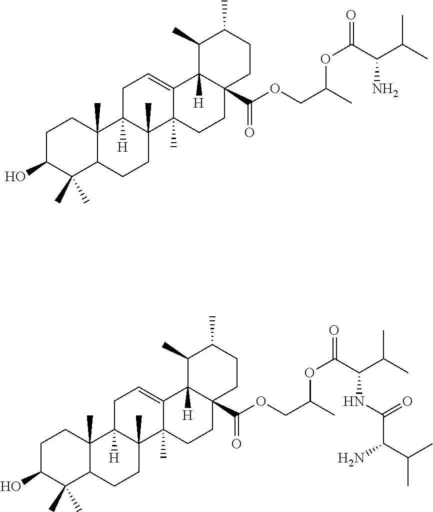
or a physiologically acceptable salt or hydrate or solvate thereof.

14. The method of claim 13, wherein the disease is an inflammatory disease, autoimmune disease, or a disease related to excessive function of Th17 cells.

15. The method of claim 13, wherein the disease is autoimmune encephalomyelitis, collagen-induced arthritis, multiple sclerosis, rheumatoid arthritis, psoriasis, Crohn's disease, asthma, inflammatory bowel disease, arthritis, melanoma, systemic lupus erythematosus, allograft rejection, ankylosing spondilitis, scleroderma, Type I diabetes, psoriatic arthritis, osteoarthritis, or atopic dermatitis.

16. The method of claim 13, wherein the composition is in a form of a tablet, a capsule, an injectable composition, an ingestible composition, a nasal spray, an aerosol, or a suppository.

17. The method of claim 13, wherein the composition is administered in a route selected from the group consisting of mucosal, oral, nasal, topical, transdermal, intradermal, parenteral, intraperitoneal, intramuscular, intravenous, subcutaneous, rectal, intraarticular, intramedullary, intraocular, buccal, and sublingual application.

18. The method of claim 13, wherein the compound is administered daily dosage of about 100 to 500 mg/kg body weight of the subject.

19. The method of claim 13, wherein the method further comprises administering to said subject a second therapeutic agent or treatment for said disease.

20. The method of claim 19, wherein said second therapeutic agent is a STAT3 inhibitor or IL-21 inhibitor.
